(19) United States
(12) Patent Application Publication
(10) Pub. No.: US 2016/0237502 A1
(43) Pub. Date: Aug. 18, 2016

(54) MARKERS FOR LIPID METABOLISM

(71) Applicant: University of Southern California, Los Angeles, CA (US)

(72) Inventor: Sean P. Curran, La Mirada, CA (US)

(73) Assignee: University of Southern California, Los Angeles, CA (US)

(21) Appl. No.: 15/050,170

(22) Filed: Feb. 22, 2016

Related U.S. Application Data
(63) Continuation-in-part of application No. 14/622,543, filed on Feb. 13, 2015.
(60) Provisional application No. 61/940,167, filed on Feb. 14, 2014.

(51) Int. Cl. C12Q 1/68; A61K 48/00; A61K 36/704; A61K 31/216; A61K 33/30; A61K 31/40; A61K 31/497; A61K 33/36; A61K 33/24; A61K 33/28; A61K 33/40; A61K 31/11

(52) U.S. Cl. CPC C12Q 1/6883; A61K 33/40; A61K 48/0083; A61K 36/704; A61K 31/216; A61K 31/11; A61K 31/40; A61K 31/497; A61K 33/36; A61K 33/24; A61K 33/28; A61K 33/30; C12Q 2600/158; C12Q 2600/118

(57) ABSTRACT

Disclosed herein is a method for treating obesity in a subject in need thereof. Also disclosed herein is a method for protecting a subject in need thereof from obesity. Further disclosed herein is a method for monitoring an efficacy of a treatment for obesity in a subject.

### CROSS-REFERENCE TO RELATED APPLICATIONS

This application is a continuation-in-part of U.S. Ser. No. 14/622,543 filed on Feb. 13, 2015, currently pending, which claims priority to U.S. Ser. No. 61/940,167, filed on Feb. 14, 2014, now expired, the contents of each of which are herein incorporated by reference in their entirety.

### STATEMENT OF GOVERNMENT INTEREST

This invention was made with government support under contract number AG032308 awarded by the National Institutes of Health. The government has certain rights in the invention.

### TECHNICAL FIELD

The present invention relates to methods for treating and diagnosing obesity, heart disease, and other diseases associated with deregulated lipid homeostasis.

### BACKGROUND

The occurrence of obesity is increasing in the human population. For example, between 1980 and 2000, obesity rates have doubled in adults and since 1980, overweight rates have doubled and tripled among children and adolescents, respectively. Obesity leads to numerous health problems, including type 2 diabetes, heart disease and other metabolic syndromes associated with deregulated lipid homeostasis.

Animals, including humans, maintain metabolic homeostasis through the coordinated metabolism of available intracellular nutrients. When fasting or starving, animals switch their utilization of dietary carbohydrates to stored fats and proteins (i.e., amino acids) to satisfy energy requirements.

Accordingly, a need remains in the art for the identification of therapeutic targets that can be manipulated to promote the use of fats as an energy source and/or prevent the accumulation of stored fat, thereby treating obesity.

### SUMMARY OF THE INVENTION

Various embodiments of the present invention provides a method of treating obesity in a subject in need thereof, the method comprising administering to the subject an agent capable of increasing activity of any one or more of Nrf2/SKN, MDT-15 or a combination thereof in the subject. In various embodiments, the agent enhances utilization of stored lipids in the subject.

Various other embodiments of the present invention provides a method of protecting a subject in need thereof from obesity, the method comprising increasing Nrf2/SKN-1 activity, MDT-15 activity, or a combination thereof in the subject. In various embodiments, the method further comprises altering the expression of a gene selected from the group consisting of fil-1, a fatty acid oxidation enzyme, and combinations thereof. In various other embodiments, the fatty acid oxidation enzyme is selected from the group consisting of acs-3, acs-11, cpt-1, cpt-3, cpt-4, cpt-5, F08A8.2, acdh-7, ech-8, GCLC, GCLM, NQO1, ACOX1, ACOX2, CPT1, CPT2, and combinations thereof.

Various other embodiments of the present invention also provides a method of monitoring the efficacy of a treatment for obesity in a subject. In various embodiments, the method comprises (a) measuring a first level of Nrf2/SKN-1 in a first sample obtained from the subject before the onset of the treatment, (b) measuring a second level of Nrf2/SKN-1 in a second sample obtained from the subject after the onset of treatment, (c) comparing the measured second level of Nrf2/SKN-1 to the measured first level of Nrf2/SKN-1; and (d) determining that the treatment is effective if the second level of Nrf2/SKN-1 is greater than the first level of Nrf2/SKN-1 or that the treatment is not effective if the second level of Nrf2/SKN-1 is equal or less than the first level of Nrf2/SKN-1. In various embodiments, the treatment is a fasting diet. In other embodiments, the method further comprises altering the expression of a gene selected from the group consisting of fil-1, a fatty acid oxidation enzyme, and combinations thereof. In yet other embodiments, the fatty acid oxidation enzyme is selected from the group consisting of acs-3, acs-11, cpt-1, cpt-3, cpt-4, cpt-5, F08A8.2, acdh-7, ech-8, GCLC, GCLM, NQO1, ACOX1, ACOX2, CPT1, CPT2, and combinations thereof.

### BRIEF DESCRIPTION OF THE FIGURES

FIG. 1A to FIG. 1I show that mutation of alh-6 enhances fat mobilization and the expression of FAO genes during starvation, in accordance with various embodiments of the present invention. (FIGS. 1a, 1b) Nile Red staining of OP50 fed wild-type and alh-6 mutants in response to fasting. The representative images are shown in FIG. 1a (scale bar, 100 μm) and quantitative data are shown in FIG. 1b (n=12 for 0 h of wild-type and alh-6 (lax105) mutants, n=13 for 3 h of alh-6 mutants, n=9 for other groups). (FIG. 1c) Expression of fil-1 in response to 3 h fasting (n=3). (FIGS. 1d,1e) Expression of FAO genes under 3 h fasted (FIG. 1d) and well-fed (FIG. 1e) conditions (n=3). (FIGS. 1f,1g) Nile Red staining of wild type and alh-6 mutants fed HT115 in response to 3 h fasting. The representative images are shown in FIG. 1f (scale bar, 100 μm) and quantitative data are shown in FIG. 1g (n=8 for fed wild type, n=9 for fed alh-6 mutants and fasted wild type, n=10 for fasted alh-6 mutants). (FIGS. 1h,1i) Survival rate of wild type and alh-6 mutant worms during starvation when fed OP50 (FIG. 1h) or HT115 (FIG. 1i) diet before starvation. Data were presented as mean±s.e.m. (*P<0.05, **P<0.01, ***P<0.001, Student's t-test, versus wild-type controls under same treatment unless specifically indicated).

FIG. 2A to FIG. 2H show that SKN-1 coordinates proline and lipid metabolism during starvation of C. elegans, in accordance with various embodiments of the present invention. (FIG. 2a) Mutation of alh-6 activates gst-4p::GFP, a SKN-1 transcriptional activity reporter, during overnight fasting. Scale bar, 100 μm. (FIG. 2b) Mutation of skn-1 abolished the activation of gst-4p::GFP. The presence of the skn-1 balancer is indicated by green fluorescent protein expression in the pharynx as pointed out by the arrow. Scale bar, 100 μm. (FIGS. 2c,2d) The increased expression of FAO genes in 3 h fasted alh-6 mutant is either dependent (FIG. 2c) or independent (FIG. 2d) on skn-1 (n=3). (FIGS. 2e,2f) Nile Red staining of worms with indicated genotypes under 3 h fasted condition. The representative images are shown in FIG. 2e (scale bar, 100 μm) and quantitative data are shown in FIG. 2f (n=11 for wild type, n=10 for other groups). (FIG. 2g) Effect of skn-1 mutation on the starvation survival rate of alh-6 mutant worms. Data were presented as mean±s.e.m. (*P<0.05, **P<0.01, ***P<0.001, Student's t-test versus controls under same treatment unless specifically indicated). (FIG. 2h) RNAi mediated knockdown of skn-1 abolished the activation of gst-4p::GFP.

FIG. 3A to FIG. 3G show that the constitutive activation of SKN-1 protects animals from HCD-induced fat accumulation, in accordance with various embodiments of the present invention. (FIGS. 3a,3b) Expression of FAO genes that are up-regulated (FIG. 3a) or down-regulated (FIG. 3b) by skn-1(lax188) gain-of-function (gof) mutation (n=3). (FIGS. 3c,3d) Nile Red staining of wild type, skn-1(lax120) and skn-1(lax188) gain-of-function mutants fed OP50 or OP50 plus 2% glucose. The representative images are shown in FIG. 3c (scale bar, 100 μm) and quantitative data are shown in FIG. 3d (n=5 for wild type fed OP50, n=10 for wild type fed OP50 plus 2% glucose, n=8 for skn-1 (lax188) fed OP50, n=7 for other groups). Data were presented as mean±s.e.m. (*P<0.05, **P<0.01, ***P<0.001, Student's t-test versus controls under same treatment unless specifically indicated). (FIGS. 3e, 3f) Knockdown of mdt-15 abolishes the activation of gst-4p::GFP in skn-1 gain of function mutants (FIG. 3e) or fasted alh-6 mutants (FIG. 3f). (FIG. 3g) Expression of FAO genes that are regulated by skn-1 (lax188) fed HT115 bacterial containing L4440 control or mdt-15 RNAi plasmids. (n=2 for skn-1 (lax188) fed control RNAi, n=3 for other groups).

FIG. 4A to FIG. 4D depict the conserved regulation of Nrf2 activity and FAO genes by Aldh4a1, in accordance with various embodiments of the present invention. (FIGS. 4a,4b) Knockdown of Nrf2 inhibits expression of its canonical target genes (FIG. 4a) and FAO genes (FIG. 4b) (n=3 for control, n=5 for Nrf2 RNAi). (FIGS. 4c,4d) Knockdown of aldh4a1 induces expression of Nrf2 target genes (FIG. 4c) (n=3) and FAO genes (FIG. 4d) (n=6). Data were presented as mean±s.e.m. (*P<0.05, **P<0.01, Student's t-test versus controls).

FIG. 5A to FIG. 5F depict that MDT-15 is a co-factor for SKN-1-mediated lipid metabolism, in accordance with various embodiments of the present invention. (FIGS. 5a,5b) RNAi mediated knockdown (FIG. 5a) or point mutation (FIG. 5b) of mdt-15 abolishes the activation of gst-4p::GFP in skn-1 gain-of-function (gof) mutants skn-1 (lax188). Scale bar, 100 μm. (FIG. 5c) Expression of FAO genes that are regulated by skn-1 (gof)-fed HT115 bacteria containing L4440 control or mdt-15 RNAi plasmids (n=2 for skn-1 (lax188)-fed control RNAi, n=3 for other groups). (FIG. 5d) The expression of alh-6-mediated FAO genes is largely dependent on mdt-15 (n=3). (FIG. 5e) Fat content of mdt-15 and alh-6; mdt-15 mutants during starvation as measured by Nile Red staining (n=13 for 0 h of mdt-15 and 16 h of alh-6; mdt-15 mutants, n=10 for 6 h of mdt-15 and alh-6; mdt-15 mutants, n=11 for 16 h of mdt-15 mutants, n=12 for 0 h of alh-6; mdt-15 mutants). (FIG. 5f) Model: during fasting, mutation of the proline catabolic gene alh-6 activates SKN-1, possibly through accumulation of metabolic intermediate P5C to mediate transcriptional program for the induction of FAO genes, which also requires co-regulator MDT-15. Constitutively activated SKN-1 induces similar transcriptional changes in FAO genes that protect animals from diet-induced obesity. Data were presented as mean±s.e.m. (*P<0.05, **P<0.01, ***P<0.001, Student's t-test versus controls under same treatment unless specifically indicated).

FIG. 6A to FIG. 6F depict the analysis of lipid metabolism in alh-6 mutants during starvation, in accordance with various embodiments of the present invention. (FIG. 6a) Schematic of amino acid catabolism pathways regulated by ALH-6, which encodes the C. elegans 1-pyrroline-5-carboxylate dehydrogenase (P5CDH). (FIG. 6b) Oil Red 0 staining of OP50 fed wild type and alh-6 mutants in response to three hours of fasting. Scale bar: 100 um. (FIG. 6c) Expression of fatty acid synthesis genes under well-fed or three hours fasting conditions (n=3). (FIGS. 6d-6e) alh-6 dependent FAO genes are either up-regulated (FIG. 6d) or down-regulated (FIG. 6e) by starvation in wild type worms (n=3). (FIG. 6f) Expression of FAO genes under three hours fasting conditions in worms fed HT115 bacteria (n=3). Data are presented as mean±SEM. (*p<0.05, **p<0.01, ***p<0.001, student's t-test, versus controls under same treatment unless specifically indicated.)

FIG. 7A to FIG. 7D depict that ROS is not involved in SKN-1 activation and lipid metabolism in fasted alh-6 mutants, in accordance with various embodiments of the present invention. (FIGS. 7a-7b), Expression of FAO genes (n=3) (FIG. 7a) and fat content as measured by Nile Red staining (n=13 for wild type and n=16 for alh-6 mutants) (FIG. 7b) in alh-6 mutants at day 3 of reproductive period. (FIG. 7c) Antioxidant NAC inhibits arsenite (AS) induced SKN-1 reporter activation but not activation when alh-6 mutants are fasted. Scale bar: 100 um. (FIG. 7d) Fat content of NAC treated worms during fasting as measured by Nile Red staining (n=8 for fasting alh-6 mutants, n=7 for other groups). Data are presented as mean±SEM. (**p<0.01, student's t-test, versus wild type controls.)

FIG. 8A to FIG. 8C depict lipid metabolism genes and steady state fat levels influenced by SKN-1, in accordance with various embodiments of the present invention. (FIG. 8a) Comparison of FAO genes that are deregulated in starved animals with compromised amino acid catabolism or well-fed animals with constitutively activated SKN-1. Genes that are increased by impaired amino acid catabolism during fasting are listed in the blue box; genes up-regulated in well-fed constitutively activated SKN-1 mutants are listed in the orange box. (FIG. 8b) Oil Red 0 staining of worms with indicated genotypes fed OP50 or OP50 supplemented with 2% glucose. Scale bar: 50 um. (FIG. 8c) Predicted SKN-1 binding sites WWTDTATC were detected within a 2 kb promoter region of each gene using Regulatory Sequence Analysis Tools (RSAT). D: Sense strand, R: antisense strand.

FIG. 9A to FIG. 9B depict the mapping of the lax225 mutation to mdt-15, in accordance with various embodiments of the present invention. (FIG. 9a) lax225 is an allele that suppresses the SKN-1 reporter activation in the skn-1 gain-of-function mutant background. Through standard SNP mapping, lax225 was linked to the center of LGIII. Further SNP mapping narrowed the genetic region between ZK121 and R01H10. 1038 RNAi clones covering this region were tested for suppression of the SKN-1 reporter activation in the skn-1 gain-of-function mutant background. A single RNAi clone targeting mdt-15 was identified. (FIG. 9b) Sequencing of mdt-15 in lax225 mutants identified a point mutation that causes a Gly to Glu change. Star: stop codon.

### DETAILED DESCRIPTION OF THE INVENTION

Unless otherwise defined, all technical and scientific terms used herein have the same meaning as commonly understood by one of ordinary skill in the art. In case of conflict, the present document, including definitions, will control. Preferred methods and materials are described herein, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The term “control sample” or “control” as used herein means a sample or specimen taken from a subject, or an actual subject who does not have enhanced lipid metabolism.

“Nucleic acids” as used herein can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine, and isoguanine. Nucleic acids can be obtained by isolation or extraction methods, by chemical synthesis methods or by recombinant methods.

A “peptide,” “protein,” or “polypeptide” as used herein can mean a linked sequence of amino acids and can be natural, synthetic, or a modification or combination of natural and synthetic.

The term “sample,” “test sample,” “specimen,” “biological sample,” “sample from a subject,” or “subject sample” as used herein interchangeably, means a sample or isolate of blood, tissue, urine, serum, plasma, amniotic fluid, cerebrospinal fluid, placental cells or tissue, endothelial cells, leukocytes, or monocytes, can be used directly as obtained from a subject or can be pre-treated, such as by filtration, distillation, extraction, concentration, centrifugation, inactivation of interfering components, addition of reagents, and the like, to modify the character of the sample in some manner as discussed herein or otherwise as is known in the art.

The term also means any biological material being tested for and/or suspected of containing an analyte of interest such as Nrf2/SKN-1 and MDT-15. The sample may be any tissue sample taken or derived from the subject. In some embodiments, the sample from the subject may comprise protein. In some embodiments, the sample from the subject may comprise nucleic acid. Any cell type, tissue, or bodily fluid may be utilized to obtain a sample. Such cell types, tissues, and fluid may include sections of tissues such as biopsy (such as muscle biopsy) and autopsy samples, frozen sections taken for histological purposes, blood (such as whole blood), plasma, serum, sputum, stool, tears, mucus, saliva, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include muscle tissue or fibers, lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary.

Methods well-known in the art for collecting, handling and processing tissue, urine, blood, serum, plasma, and other body fluids, are used in the practice of the present disclosure. The test sample can comprise further moieties in addition to the analyte of interest, such as antibodies, antigens, haptens, hormones, drugs, enzymes, receptors, proteins, peptides, polypeptides, oligonucleotides or polynucleotides. For example, the sample can be a whole blood sample obtained from a subject. It can be necessary or desired that a test sample, particularly whole blood, be treated prior to immunoassay as described herein, e.g., with a pretreatment reagent. Even in cases where pretreatment is not necessary (e.g., most urine samples, a pre-processed archived sample, etc.), pretreatment of the sample is an option that can be performed for mere convenience (e.g., as part of a protocol on a commercial platform). The sample may be used directly as obtained from the subject or following pretreatment to modify a characteristic of the sample. Pretreatment may include extraction, concentration, inactivation of interfering components, and/or the addition of reagents.

The term “subject” or “patient” as used herein interchangeably, means any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc)) and a human. In some embodiments, the subject or patient may be a human or a non-human. The subject or patient may be undergoing other forms of treatment.

“Treat”, “treating” or “treatment” are each used interchangeably herein to describe reversing, alleviating, or inhibiting the progress of a disease, or one or more symptoms of such disease, to which such term applies. “Treatment” and “therapeutically,” refer to the act of treating, as “treating” is defined herein. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of an antibody or pharmaceutical composition of the present invention to a subject that is not at the time of administration afflicted with the disease. “Preventing” also refers to preventing the recurrence of a disease or of one or more symptoms associated with such disease. In some embodiments, “protecting” refers to the prevention of disease in a subject. For example, protecting a subject from obesity may include a subject who is predisposed to metabolic disease, an obesity related condition and/or a genetic condition that predisposes the subject to obesity. A “predisposed subject” refers to an individual who is determined to be more likely to develop a symptom based on conventional risk assessment methods or has one or more risk factors that correlate with development of obesity. A predisposed subject may or may not have detectable disease, and may or may not have displayed detectable disease prior to the treatment methods described herein.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence/s substantially identical thereto.

Variant can further be defined as a peptide or polypeptide that differs in amino acid sequence by the insertion, deletion, or conservative substitution of amino acids, but retain at least one biological activity. Variants can be a fragment thereof. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retains protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Various embodiments of the present invention provides for a method of treating obesity in a subject in need thereof, the method comprising administering to the subject an agent capable of increasing Nrf2/SKN-1 activity, MDT-15 activity, or a combination thereof in the subject. In various embodiments, the agent enhances utilization of stored lipids in the subject.

Various other embodiments of the present invention provides for a method of protecting a subject in need thereof from obesity, the method comprising increasing Nrf2/SKN-1 activity, MDT-15 activity, or a combination thereof in the subject. In various embodiments, the method further comprises altering the expression of a gene selected from the group consisting of fil-1, a fatty acid oxidation enzyme, and combinations thereof. In various other embodiments, the fatty acid oxidation enzyme is selected from the group consisting of acs-3, acs-11, cpt-1, cpt-3, cpt-4, cpt-5, F08A8.2, acdh-7, ech-8, GCLC, GCLM, NQO1, ACOX1, ACOX2, CPT1, CPT2, and combinations thereof.

Various other embodiments of the present invention also provides for a method of monitoring the efficacy of a treatment for obesity in a subject. In various embodiments, the method comprises (a) measuring a first level of Nrf2/SKN-1 in a first sample obtained from the subject before the onset of the treatment, (b) measuring a second level of Nrf2/SKN-1 in a second sample obtained from the subject after the onset of treatment, (c) comparing the measured second level of Nrf2/SKN-1 to the measured first level of Nrf2/SKN-1; and (d) determining that the treatment is effective if the second level of Nrf2/SKN-1 is greater than the first level of Nrf2/SKN-1 or that the treatment is not effective if the second level of Nrf2/SKN-1 is equal or less than the first level of Nrf2/SKN-1. In various embodiments, the treatment is a fasting diet. In other embodiments, the method further comprises altering the expression of a gene selected from the group consisting of fil-1, a fatty acid oxidation enzyme, and combinations thereof. In yet other embodiments, the fatty acid oxidation enzyme is selected from the group consisting of acs-3, acs-11, cpt-1, cpt-3, cpt-4, cpt-5, F08A8.2, acdh-7, ech-8, GCLC, GCLM, NQO1, ACOX1, ACOX2, CPT1, CPT2, and combinations thereof.

### Method of Identifying a Factor for Enhancing Lipid Metabolism

Provided herein is a method of identifying factors and sub-factors for enhancing lipid metabolism in a subject in need thereof. The method includes obtaining a sample from the subject and measuring or detecting a level of the factor in the sample either alone or in combination with one, two, three, or more factors. The method also includes measuring or detecting a level of a sub-factor in the sample alone, in combination with the factor, in combination with one, two, three or more factors, in combination with one, two, three, or more sub-factors, or any combination thereof.

In various embodiments, a change in the level of the factor in the sample obtained from the subject relative to a control sample identifies the factor for enhancing lipid metabolism, thereby indicating that lipid metabolism is enhanced or increased in the subject. In some embodiments, the change in the level of the factor may be an increase in or an up-regulation of the expression or activity of the factor in the sample obtained from the subject. In other embodiments, the change in the level of the factor may be a decrease in the level of or an absence of the factor in the sample obtained from the subject. In yet other embodiments, the change in the level of the factor may be a decrease or a down-regulation of the expression or activity of the factor in the sample obtained from the subject.

In various other embodiments, a change in the level of the sub-factor in the sample obtained from the subject relative to the control sample identifies the sub-factor for enhancing lipid metabolism, thereby indicating that lipid metabolism is enhanced or increased in the subject. In some embodiments, the change in the level of the sub-factor may be an increase in or an up-regulation of the expression or activity of the sub-factor in the sample obtained from the subject. In other embodiments, the change in the level of the sub-factor may be a decrease in the level of or an absence of the sub-factor in the sample obtained from the subject. In yet other embodiments, the change in the level of the sub-factor may be a decrease or a down-regulation of the expression or activity of the sub-factor in the sample obtained from the subject.

In some embodiments, the method can identify one, two, three, or more factors for enhancing lipid metabolism alone or in combination in the sample obtained from the subject in need thereof. In other embodiments, the method can measure or detect the change in the level of the factor in the sample alone, in combination with one, two, three, or more factors, in combination with one, two, three, or more sub-factors, or any combination thereof.

In various embodiments, the factor may be a nucleic acid sequence, an amino acid sequence, or a combination thereof. In various other embodiments, the nucleic acid sequence may be DNA, RNA, cDNA, a variant thereof, a fragment thereof, or a combination thereof. The amino acid sequence may be a protein, a peptide, a variant thereof, a fragment thereof, or a combination thereof.

In various embodiments, the factor may be Nrf2/SKN-1. SKN-1 is the C. elegans ortholog of mammalian Nrf2. Nrf2/SKN-1 is a transcription factor that regulates oxidative stress responses and longevity. Gain-of-function mutations in SKN-1 may induce a starvation-like state. As described herein, Nrf2/SKN-1 may also regulate lipid metabolism. In some embodiments, Nrf2/SKN-1 regulation of lipid metabolism may be mediated by or dependent upon MDT-15, described herein. In yet other embodiments, Nrf2/SKN-1 may regulate lipid metabolism by up-regulating the depletion and/or mobilization of stored fat or lipids.

In various embodiments, Nrf2/SKN-1 may promote usage of stored fats in a diet-dependent manner. In various other embodiments, Nrf2/SKN-1 may promote usage of stored fats in response to food deprivation such as fasting and starvation. In other embodiments, Nrf2/SKN-1 may promote usage of stored fats in the presence of a high carbohydrate diet. In yet other embodiments, Nrf2/SKN-1 may promote usage of stored fats when amino acid catabolism is repressed or otherwise inactivated.

In various other embodiments, Nrf2/SKN-1 activity (e.g., constitutive activity) may prevent or decrease accumulation of fat. In yet other embodiments, Nrf2/SKN-1 activity may prevent or decrease accumulation of fat in the presence of a high carbohydrate diet.

In various embodiments, the factor may be MDT-15. MDT-15 is a transcriptional regulator of lipid metabolism and interacts with Nrf2/SKN-1. In various embodiments, the method can identify one, two, three, or more sub-factors alone, in combination, or in combination with the factors described herein. In some embodiments, the method may measure or detect the change in the level of the sub-factor alone, in combination with one, two, three, or more sub-factors, in combination with one, two, three, or more factors, or any combination thereof.

In various embodiments, the sub-factor may be a nucleic acid sequence, an amino acids sequence, or a combination thereof. In other embodiments, the nucleic acid sequence may be DNA, RNA, cDNA, a variant thereof, a fragment thereof, or a combination thereof. In some embodiments, the amino acid sequence may be a protein, a peptide, a variant thereof, a fragment thereof, or a combination thereof.

In various other embodiments, the sub-factor may be fasting-induced lipase-1 (fil-1). Fil-1 levels may be increased in the sample obtained from the subject relative to the control sample, thereby identifying fil-1 as a sub-factor for enhancing lipid metabolism in the subject. Fil-1 mRNA levels may be increased about 1.2-fold to about 4.0-fold, or about 1.5-fold to about 3.0-fold, or about 2-fold in the sample obtained from the subject. In other embodiments, fil-1 mRNA levels may be increased about 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, or 4.0-fold in the sample obtained from the subject.

In various other embodiments, the sub-factor may be a fatty acid oxidation (FAO) enzyme. Under fasting conditions, fat is utilized through the activation of mitochondrial and peroxisomal fatty oxidation (FAO). These FAO pathways are mediated by a number of enzymes, namely FAO enzymes. In yet other embodiments, the FAO enzyme may be acs-3, acs-11, cpt-1, cpt-3, cpt-4, cpt-5, F08A8.2, acdh-7, ech-8, GCLC, GCLM, NQO1, ACOX1, ACOX2, CPT1, or CPT2.

In one embodiment, an mRNA level of one or more of the FAO enzymes, acs-1, acs-3, acs-11, cpt-1, cpt-3, cpt-4, cpt-5, cpt-6, acox-1, F08A8.2, F08A8.3, F08A8.4, acdh-1, acdh-7, B0272.3, ech-1, ech-4, B0303.3, F53A2.7, and ech-8 may be increased about 1.2-fold to about 4.0-fold or about 1.5-fold to about 3.0-fold in the sample obtained from the subject. In other embodiments, the mRNA level of one or more of the FAO enzymes acs-1, acs-3, acs-11, cpt-1, cpt-3, cpt-4, cpt-5, cpt-6, acox-1, F08A8.2, F08A8.3, F08A8.4, acdh-1, acdh-7, B0272.3, ech-1, ech-4, B0303.3, F53A2.7, and ech-8 may be increased about 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, or 4.0-fold in the sample obtained from the subject.

In still other embodiments, the increased mRNA level of one or more of the FAO enzymes, acs-3, acs-11, cpt-1, cpt-3, cpt-4, acdh-7, and ech-8 may be dependent upon Nrf2/SKN-1, which is described herein. In other embodiments, the increased mRNA level of cpt-5 and F08A8.2 may not be dependent upon Nrf2/SKN-1.

In other embodiments, the mRNA level of one or more FAO enzymes acs-18, C48B4.1, acdh-2, acdh-8, hacd-1, ech-7, and ech-9 may be decreased by about 10% to about 90% or about 25% to about 75% in the sample obtained from the subject. In other embodiments, the mRNA level of one or more FAO enzymes, acs-18, C48B4.1, acdh-2, acdh-8, hacd-1, ech-7, and ech-9 may be decreased by about 10%, 15%, 20%, 25%, 30%, 35%, 40%, 45%, 50%, 55%, 60%, 65%, 70%, 75%, 80%, 85%, or 90% in the sample obtained from the subject.

In some embodiments, expression of GCLC, GCLM, NQO1, ACOX1, ACOX2, CPT1, and CPT2 may be dependent upon Nrf2/SKN-1 in mammalian cells, for example, human cells.

Method of Treatment and/or Protection

Also provided herein is a method for treating a disease in a subject in need thereof, the method comprising administering to the subject an agent capable of increasing Nrf2/SKN-1 activity, MDT-15 activity, or a combination thereof in the subject. In one embodiment, the disease is obesity.

In various embodiments, administering the agent may treat disease (for example, obesity) in a subject in need thereof. In various other embodiments, administering the agent alters (e.g., increases) the level or activity of one or more of the factors described herein and may treat the disease in the subject. In various other embodiments, administering the agent alters the level or activity of one or more of the sub-factors described herein and may treat the disease in the subject. In some embodiments, the disease may be obesity, heart disease, or a metabolic disorder associated with deregulated lipid homeostasis. In some embodiments, the disease may be obesity.

Also provided herein is a method for protecting a subject in need thereof from a disease, the method comprising administering to the subject an agent capable of increasing Nrf2/SKN-1 activity, MDT-15 activity, or a combination thereof in the subject. In one embodiment, the disease is obesity.

In various embodiments, administering the agent may protect the subject from the disease (for example, obesity). In various other embodiments, administering the agent alters (e.g., increases) the level or activity of one or more of the factors described herein and may protect the subject from the disease. In yet other embodiments, administering the agent alters the level or activity of one or more of the sub-factors described herein and may protect the subject from the disease. In some embodiments, the disease may be obesity, heart disease, or a metabolic disorder associated with deregulated lipid homeostasis. In other embodiments, the disease may be obesity.

In various embodiments, the factors and/or sub-factors, as described herein, may be directly or indirectly altered by the agent being administered.

In various other embodiments, administering the agent to the subject suffering from the disease, may alter the level or activity of one or more of the factors, discussed herein, such that the level or activity leads to increased utilization of lipids, fat, stored lipids, and/or stored fat and/or prevents accumulation of lipids and/or fat in the subject thereby treating and/or preventing the disease. In various embodiments, the agent may enhance utilization of stored lipids or fat in the subject. In some embodiments, the disease may be obesity, heart disease, or a metabolic disorder associated with deregulated lipid homeostasis. In other embodiments, the disease may be obesity.

In other embodiments, administering the agent to the subject suffering from the disease, may alter the level or activity of one or more of the sub-factors, discussed herein, such that the level or activity leads to increased utilization of lipids, fat, stored lipids, and/or stored fat and/or prevents accumulation of lipids and/or fat in the subject thereby treating and/or preventing the disease. In some embodiments, the agent may enhance utilization of stored lipids or fat in the subject. In some embodiments, the disease may be obesity, heart disease, or a metabolic disorder associated with deregulated lipid homeostasis. In other embodiments, the disease may be obesity.

In various embodiments, potential agents that can be used to alter the level or activity of one or more of the factors and/or sub-factors, include, but are not limited to those listed in Table 1.

TABLE 1
Nrf2 Activators
Nrf2 Activators

Roots of Polygonum cuspidatum (PC)
Ebselen
caffeic acid phenethylester
cinnamic aldehyde
bardoxolone methyl
Nrf2 Activator (from Xymogen)
Ultimate Protector: NRF2 Activator (from Integrated Health)
Triterpenoids:
CDDO-Me
CDD-IM
Oxidisable diphenols, phenylenediamines, Quinones:
BHA: (2(3)-tert-butyl-4-hydroxyanisole)
tBHQ: tert-butylhydroquinone
Michael reaction acceptors:
Curcumin (Tumeric)
CDDO-Me
Zerumbone (ginger)
Citral (plant oils)
Isothiocyanates and sulfoxythiocarbamates:
Benzyl isothiocyanate
Sulforphane (Sfn)
Thiocarbamates:
Pyrrolidine dithiocarbamate (PDTC)
Dithiolethiones:
Oltipraz: 4-methyl-5-pyrazinyl-3H-1,2-dithiole-3-thione
R-lipoic acid
Polyenes:
Chlorophyll
Porphyrins
Chlorophyllins
Hydroperoxides:
Hydrogen Peroxide
Trivalent arsenicals:
Arsenic Trioxide
Heavey metals:
Methyl Mercury
Cadmium
Zinc
Dimercaptans:
Mercaptan

### Method of Monitoring Efficacy of Treatment

Also provided herein is a method of monitoring the efficacy of treatment of the disease in a subject undergoing treatment of the disease in any form. In some embodiments, the treatment may be a fasting diet. In various embodiments, monitoring for efficacy may entail applying the method of identifying factors and/or sub-factors for enhancing lipid metabolism described herein, to determine if the treatment of the disease has a therapeutic effect in the subject. In various embodiments, the disease may be obesity, heart disease, or a metabolic disorder associated with deregulated lipid homeostasis. In various other embodiments, the disease may be obesity.

In some embodiments, monitoring for efficacy may include obtaining a first sample from the subject before treatment has begun and a second sample from the subject after treatment has begun. In various embodiments, the levels of one or more factors can be measured or detected in the first and second samples to determine a first level and a second level of the one or more factors. In various other embodiments, the first and second levels of the one or more factors measured may be compared to determine if the second level is different or changed (e.g., higher or lower) from the first level. In some embodiments, a difference in the measured level of the factor may indicate whether the disease treatment has had a therapeutic effect in the subject.

In various embodiments, the method of monitoring may also include measuring or detecting a first and second level of one or more sub-factors in the first and second samples, and comparing the first and second levels of the one or more sub-factors. In various other embodiments, the first and second levels of the one or more sub-factors measured may be compared to determine if the second level is different or changed (e.g., higher or lower) from the first level. In some embodiments, a difference in the measured level of the sub-factor may indicate whether the disease treatment has had a therapeutic effect in the subject.

In some embodiments, the method of monitoring may include monitoring the efficacy of a treatment for obesity in the subject. In some embodiments, the method may include measuring a first level of Nrf2/SKN-1 in the first sample obtained from the subject before the onset of treatment and measuring a second level of Nrf2/SKN-1 in the second sample obtained from the subject after the onset of treatment. In other embodiments, the method may also include comparing the measured second level of Nrf2/SKN-1 to the measured first level of Nrf2/SKN-1 and determining if the treatment is effective if the second level of Nrf2/SKN-1 is greater than the first level of Nrf2/SKN-1 or that the treatment is not effective if the second level of Nrf2/SKN-1 is equal to or less than the first level of Nrf2/SKN-1.

### Kit

Also provided herein is a kit for use with the methods disclosed herein. In various embodiments, the kit can include one or more reagents for detecting the factors and sub-factors either alone or in any combination thereof. In some embodiments, the one or more reagents may be any of those reagents known in the art for immunoassays (e.g., ELISA, western blotting, immunoprecipitation (IP), immunohistochemistry, etc.) used to detect the factors and sub-factors. In other embodiments, the one or more reagents can also be any of those reagents known in the art for detecting nucleic acids, for example, but not limited to, polymerase chain reaction (PCR), reverse-transcriptase-PCR (RT-PCR), northern blotting, quantitative PCR (qPCR), and so forth. In other embodiments, the kit may also include one or more controls and instructions for how to use the kit.

The present invention has multiple aspects, illustrated by the following non-limiting examples. It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

### EXAMPLES

### Example 1

### Materials and Methods

C. elegans Growth Conditions and Strains.

C. elegans were cultured using standard techniques at 20° C. (Brenner, Genetics; 77, 71-94; 1974). The following strains were used: wild-type N2 Bristol, SPC207: skn-1 (lax120), SPC227: skn-1 (lax188), SPC321: alh-6 (lax105), CL2166: gst4-p::gfp, SPC276: skn-1 (lax188); mdt-15 (lax225); gst4-p::gfp, VC1772: skn-1 (ok2315) IV/nTi[qIs51] (IV; V) and XA7702: mdt-15 (tm2182). Double or triple mutants were generated by standard genetic techniques. Animals are fed on E. coli OP50 or RNAi inducing bacterial E. coli HT115.

Starvation Assay.

For starvation, synchronized L1 animals were added to nematode growth medium (NGM) plates seeded with indicated bacteria. After 2 days at 20° C., L4 animals were collected, washed with M9 buffer at least three times and then subjected to fasting in M9 liquid with shaking for indicated time before collection for further analysis. Starvation survival assay was performed as previous described (Artyukhin et al., Sci. Rep.; 3, 2777; 2013). Briefly, gravid worms that did not experience starvation for at least two generations were used for egg preparation. After 24 h, synchronized L1 animals were resuspended in M9 at a concentration of two worms per microliter. Starvation culture was mixed by constant rocking. Every 2 days, a portion of animals was recovered on normal OP50-seeded NGM plates. Animals that resumed development were considered to be surviving.

Nile Red Staining.

Nile Red staining was performed as previously described (Pino, et al., J. Vis. Exp. 73; 2013). Briefly, animals of indicated genotypes were collected, fixed in 40% isopropanol at room temperature for 3 min and stained in 3 μg ml-1 Nile Red working solution in dark for 2 h. Worms were then washed with M9 for at least 30 min, mounted on slides and imaged under the green fluorescent protein channel of microscope Zeiss Axio Imager with Zen software package. Fluorescent density was measured using ImageJ software. Approximately ten animals from each experiment (n) were used to calculate the fluorescent density.

Oil Red O Staining.

Animals of indicated genotypes were collected and fixed in 1% formaldehyde in PBS for 10 min. Next, samples were frozen and thawed three times with dry ice/ethanol bath. Worms were washed with PBS three times before staining with freshly prepared Oil Red O working solution. Worms were stained while rotating for 30 min, washed again with PBS for 15 min, mounted on slides and imaged under a bright-field illumination.

RNAi Treatment.

HT115 bacteria containing specific double stranded RNA-expression plasmids were seeded on NGM plates containing 5 mM isopropyl-β-D-thiogalactoside and 50 μg ml-1 carbenicillin. RNAi was induced at room temperature for 24 h. Synchronized L1 animals were added to those plates to knockdown indicated genes.

Quantitative Reverse Transcription-PCR.

Quantitative reverse transcription-PCR was performed as previously described (Pang et al., Cell Metab. 19, 221-231; 2014). Briefly, worms of the indicated genotype and stages were collected, washed in M9 buffer and then homogenized in Trizol reagent (Life Technologies). RNA was extracted according to the manufacturer's protocol. DNA contamination was digested with DNase I (New England Biolabs) and subsequently RNA was reverse-transcribed to complementary DNA by using the SuperScript III First-Strand Synthesis System (Life Technologies). Quantitative PCR was performed by using SYBR Green (BioRad). The expression levels of snb-1 and actin were used to normalize samples in worms and human cells, respectively. Primer sequences listed in Table 2.

TABLE 2
qPCR primer sequences.

| C. | | SEQ | | SEQ |
|---|---|---|---|---|
| elegans | | ID | | ID |
| Genes | Forward Sequences | NO. | Reverse Sequences | NO. |
| acs-2 | AAGGAGATGAGAATGAC | 1 | GTTCCGACATGGTGACTA | 2 |
| | TGAT | | | |
| acs-3 | CACGATTCAAGCAACTTC | 3 | TTCACTTCCTTATTCTCCAT | 4 |
| | | | TA | |
| acs-11 | GCTTATTGGAATTATGAA | 5 | GGACCTTAGTGATGTGAT | 6 |
| | GAAG | | | |
| acs-13 | ATCAGGCAGAGATCAAGA | 7 | GGTTCCATCACAACAAGT | 8 |
| acs-18 | GCTTACAATGTCCTATCG | 9 | TCCATCTTCTTGAATAATCG | 10 |
| RO9E10.4 | ATTGATACTGGCGATGAA | 11 | AGAGATTGGTTATGTAATG | 12 |
| | | | C | |
| cpt-1 | TCTATTGTCGTGGAGTCT | 13 | GGATTGCGTCGTATTGTA | 14 |
| cpt-3 | TACACGGATTCTATGAAGT | 15 | TAGTTGTCTGTGATTAGGT | 16 |
| cpt-4 | CCAGCACTTCAGGATACT | 17 | AGCAGTTGGTCATAGTCTT | 18 |
| cpt-5 | GACAGCACAATTCGTAG | 19 | TCTCCAGCCAACATATCT | 20 |
| | TA | | | |
| cpt-6 | CACTTCTACTTCTCATACA | 21 | TCACAAGATTCAAGGATT | 22 |
| acdh-1 | CTCTGTTCTGATAGTCTT | 23 | CCTCTCCTGAATTAGTAA | 24 |
| acdh-2 | CAGGAACCATTGCTCAAG | 25 | ATTCAGAACTTCAATAGCG | 26 |
| | | | TAT | |
| acdh-3 | CACAAGTCGTCAATTCTG | 27 | TGAGCCAATCCTAACATT | 28 |
| acdh-7 | ACCAAGTGTGAGAAGAAG | 29 | CGAAGAACCAGTTAGCAT | 30 |
| acdh-8 | AATCATCAAGGAGTTCA | 31 | AAGAGACACCATAAGAG | 32 |
| | AT | | T | |
| acdh-9 | TTCTGTCTTATGATTGAG | 33 | CTTGTTGGTTGTGAGTTC | 34 |
| | GAT | | | |
| acdh- 12 | AACTCTTGGAATGGTGA | 35 | AACTGCTCTTGGAATGTC | 36 |
| | TG | | | |
| ech-1 | CGAATGTAACTATCAATA | 37 | ATGGCGGAATAATCAATT | 38 |
| | AGG | | | |
| ech-2 | GAGTTGAAGGCTATTGAC | 39 | ATATTGCGGATGAAGTTC | 40 |
| ech-4 | AACGCATTGACATTGGAA | 41 | CATTGGCAGTAATCACAGT | 42 |
| ech-5 | GTGTGGTGATTCTCAACTC | 43 | CTGCTGGCTCATAGTCTT | 44 |
| ech-6 | TGGATACTGATAAGTCTGT | 45 | CTCGTTATTGGTCATCTC | 46 |
| ech-7 | TGTTCTGTTGGCTGATAG | 47 | TTTAGGCTGGTTTGGTAG | 48 |
| hacd-1 | GCTTGTAGAAGTTGTATC | 49 | AATGAATCCAGGAGTATC | 50 |
| B0272.3 | TGTGGATAGCAATCAAT | 51 | CTTCTTCTTGGCAACTCTC | 52 |
| | CTG | | | |
| F54C8.1 | GATAGTCCTGGATTCAT | 53 | GCATCTCCTCGTTCATAC | 54 |
| | TGT | | | |
| T08B2.7 | TGCTCAGGAACTTGCTAA | 55 | GTCATAACTGCTTGCGTAG | 56 |
| F53A2.7 | AAGTTACCAGACAAGAAG | 57 | ATAGTGATTCCAACGATT | 58 |
| B0303.3 | GAAGAGAACAAGACGA | 59 | TAAGAATACTGGAACAA | 60 |
| | AT | | CAT | |
| acox-1 | CTTCAACAACTACCGTAT | 61 | AGCATATAACTTCTCACAT | 62 |
| F08A8.2 | ATCTAACCAGCCTGAATG | 63 | ATGCCACTTCTCTTGATT | 64 |
| F08A8. 3 | ACGATACAATGTTCATAC | 65 | AATAATCTCCTTCCTTCTA | 66 |
| F08A8.4 | CAGCAGCATCGTCTATTC | 67 | GTTCAATACCTTCTCCAGTT | 68 |
| F59F4.1 | ACTCACTATCCACTCAAG | 69 | CGTATCTCATCAGCAATG | 70 |
| C48B4.1 | CTCTGATGTTCTTGCTGAT | 71 | ATGCTTGTCTTGCTTGAT | 72 |
| ech-3 | GAGCACTGGATATGATAC | 73 | CCGAGATTCTATTGACAA | 74 |
| ech-8 | GAGGAAGGAATCATCCA | 75 | GACCACCAGTAGCAACAG | 76 |
| | TCAT | | | |
| ech-9 | GTATTCAACAGGCTTCTT | 77 | CCATTCATATCGGCAACA | 78 |
| | CAT | | G | |
| T02G5. 7 | ACTATTCTTGTTGTTGGA | 79 | GCATCTTGTTCTTCTCTA | 80 |
| fil-1 | GTGTCAACTATTCCTCATT | 81 | GGTTCCGTATTCAATAAGA | 82 |
| | | | T | |
| | | SEQ | | SEQ |
| Human | | ID | | ID |
| Genes | Forward Sequences | NO. | Reverse Sequences | NO. |
| Cpt1 | AATAAGCAGTCTCTTGA | 83 | CACTTCTGTATCCTTCTT | 84 |
| | TG | | C | |
| Cpt2 | TCTACTTCTTAGGTGAGG | 85 | CGTGATTGGAATCTGATA | 86 |
| | AA | | AC | |
| Acox1 | GCAGCCAGATTAGTAGAA | 87 | AACAAGGTCAACAGAAGT | 88 |
| Acox2 | CACTTGGCTGTTATGATG | 89 | GTTCTCCTGAGTATTGGT | 90 |
| Acads | GATTGTGCTGTGAACTAC | 91 | CAACTTGAACTGGATGAC | 92 |
| Acadm | CTGGTGCTGTTGGATTAG | 93 | ATATTGCTTGGTGCTCTAC | 94 |
| Acadl | TAGTATTCATTCAGGTAT | 95 | GCTCTGTCATTGCTATTG | 96 |
| | TGTC | | | |
| Ech | TAGAGTGCTTCAACAAGA | 97 | ATGTCCATCAGGTCAATA | 98 |
| Hadha | CCGTCCTTATCTCATCAA | 99 | AACTATTCTCTGTGCTTCT | 100 |
| Hadhb | ACACTCACACTAGGCAA | 101 | AACACTGGCAAGGCTTAA | 102 |
| | TG | | | |
| Nrf2 | ACACGGTCCACAGCTCAT | 103 | TTGACATACTTTGGAGGCA | 104 |
| | C | | AGA | |
| Gclc | CAAGGACGTTCTCAAGT | 105 | AGAGAAGGGGGAAAGGA | 106 |
| | GGG | | CAA | |
| Gclm | CTGTGTGATGCCACCAG | 107 | TGAAGCAAGTTTCCAAGA | 108 |
| | ATT | | AGC | |
| Nqo1 | GGACTGCACCAGAGCCAT | 109 | CGGCTTTGAAGAAGAAAGG | 110 |
| | | | A | |

Human Cell Culture.

293T cells were cultured in DMEM supplemented with 10% fetal bovine serum. At 50-70% confluence, cells were transfected with control, Nrf2/Nfe212(s9492) or aldh4a1(s16484) Silencer Select (Life Technologies) siRNA by using Lipofectamine RNAiMax (Life Technologies). After 24 h, cells were washed and collected in Trizol reagent (Invitrogen) for further RNA extraction. For examining the Nrf2 target genes in aldh4a1 siRNA experiment, cells were collected 48 h after transfection.

Statistical Analysis.

Data are presented as mean±S.E.M. Data were analyzed by using unpaired Student's t-test. P<0.05 was considered as significant.

### Example 2

Animals maintain metabolic homeostasis through the coordinated metabolism of available intracellular nutrients. Animals maintain energy homeostasis through the coordinated metabolism of available intracellular nutrients, including glucose, lipids and amino acids. To do so, animals employ complex but elegant molecular mechanisms to integrate the metabolism of these nutrients. In mice for example, under well-fed conditions, the liver X receptor integrates hepatic glucose metabolism and lipogenesis by acting as a glucose sensor. Glucose-mediated ChREBP activation in adipose tissue activates fatty acid synthesis, thus connecting glucose and lipid metabolism. Although mechanisms such as these linking glucose and lipid metabolism have been well recognized, it remains largely elusive whether and how the metabolism of amino acids and lipids, two major nutrients for fasting responses, are coordinated.

During periods of nutrient deprivation, stored lipids and amino acids are used instead of dietary glucose to satisfy organismal energy requirements. Lipids are mobilized as an energy resource through lipolysis and fatty acid oxidation (FAO). Meanwhile, amino acids, another important energy resource, are catabolized as fuel during starvation. In particular, proline, arginine and ornithine are catabolized through common metabolic pathways and are preferentially utilized by animals in response to acute food deprivation, as evidenced by their early and significant reduction under fasting conditions. Amino Acids can either be directly oxidized or converted to glucose, and then oxidized by organs with an obligatory glucose requirement. Based on the universal importance of these metabolic pathways during starvation, it is possible that their metabolism may be coupled together during fasting, and that this link would be well conserved.

SKN-1 is the worm homologue of the mammalian transcription factor Nrf2, both of which share a conserved cytoprotective function in the response to cellular electrophiles. In addition, SKN-1 is a well-known longevity factor that is activated in many long-lived mutant backgrounds with altered metabolic homeostasis and is indispensable for the lifespan extension of those mutants. Recently, we reported that gain-of-function mutations in skn-1 lead to a starvation-like status in C. elegans and induce the expression of several metabolic genes; however, the full extent to which SKN-1 participates in organismal physiology and metabolism remains unknown.

C. elegans is an established model for studying conserved pathways that govern lipid metabolism. In this study, by using a C. elegans strain with a mutation in a conserved proline catabolic gene, we investigate the role of proline catabolism in the organismal response to fasting and discover that proline catabolism is coupled with fasting lipid utilization by the transcription factor SKN-1/Nrf2.

This study demonstrates that during starvation the metabolism of amino acids and lipids are transcriptionally coordinated by SKN-1/Nrf2. In response to fasting, C. elegans with compromised amino acid catabolism display accelerated mobilization of stored fat and enhanced expression of genes in fatty acid oxidation. Notably, this phenotype is dependent on the type of food ingested prior to fasting indicating that diets can predetermine future metabolic adaption responses during food deprivation. Defective amino acid catabolism when coupled to starvation leads to hyper SKN-1 activation, which mediates this lipid metabolism response. Constitutive SKN-1 activation is capable of inducing a similar transcriptional response, which remarkably protects animals from fat accumulation when fed a high carbohydrate diet. In human cells, Nrf2 activity is similarly coupled to the expression of fatty acid oxidation genes. These findings identify a novel mechanism for coordinating the metabolism of lipids and amino acids, and implicate SKN-1/Nrf2 as a potential therapeutic target for obesity.

### Example 3

### Loss of Alh-6 Accelerates Lipid Mobilization During Fasting

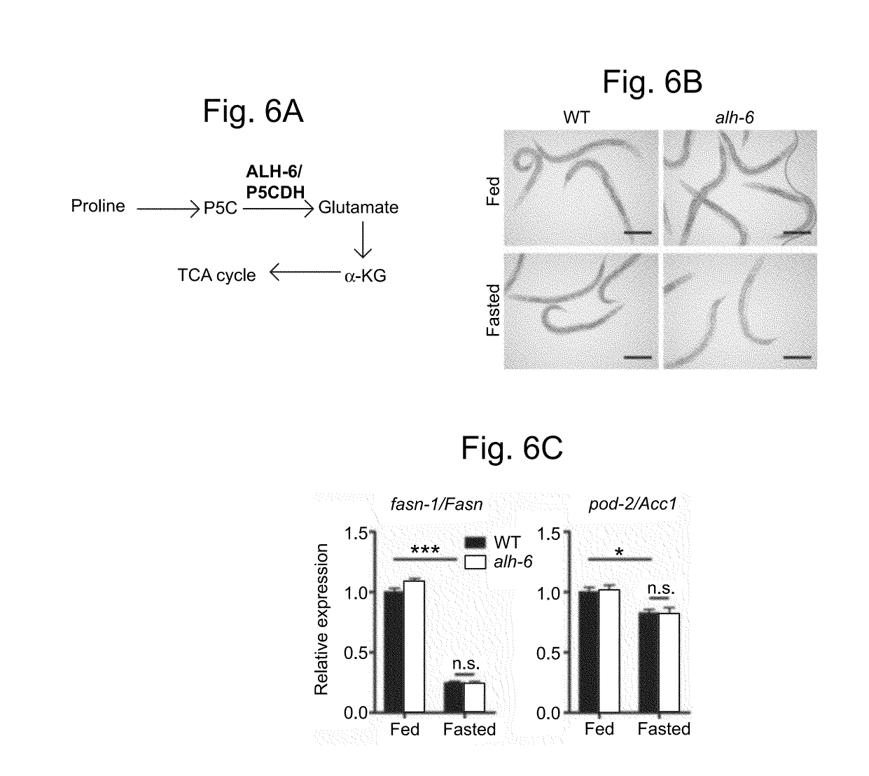

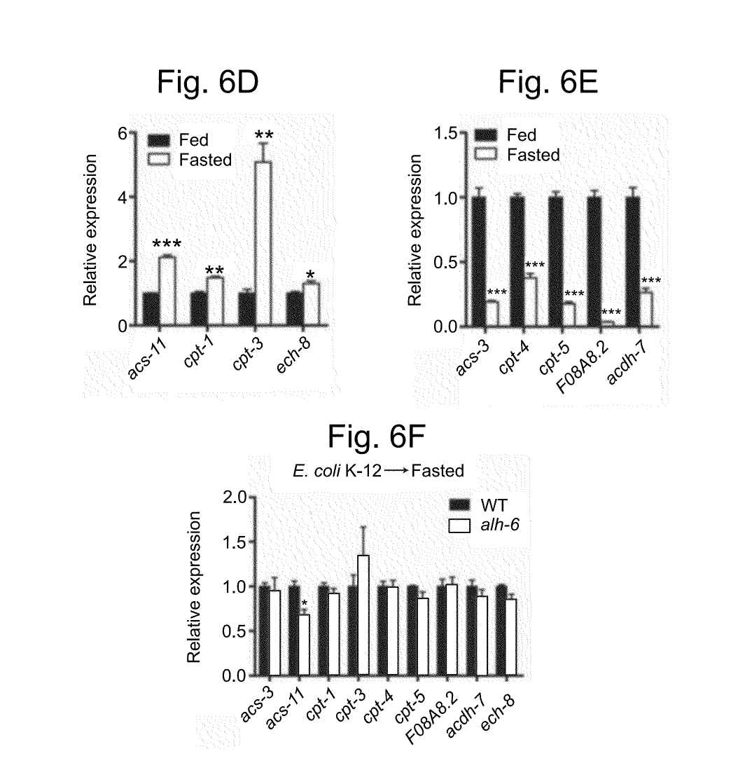

In a previous study, we identified mutations in the C. elegans gene alh-6, which is an evolutionarily conserved mitochondrial enzyme involved in the catabolism of proline and encodes the C. elegans 1-pyrroline-5-carboxylate dehydrogenase (P5CDH) (FIG. 6a). Thus, the alh-6 mutation represents a metabolic state defined by compromised catabolism of amino acids central for the fasting response. While not being bound to any particular theory, it is hypothesized that during starvation the use of amino acids and lipids are functionally linked, thus prompting an investigation of the effects of alh-6 mutations on fat metabolism. C. elegans is an established model for studying conserved pathways that govern lipid metabolism.

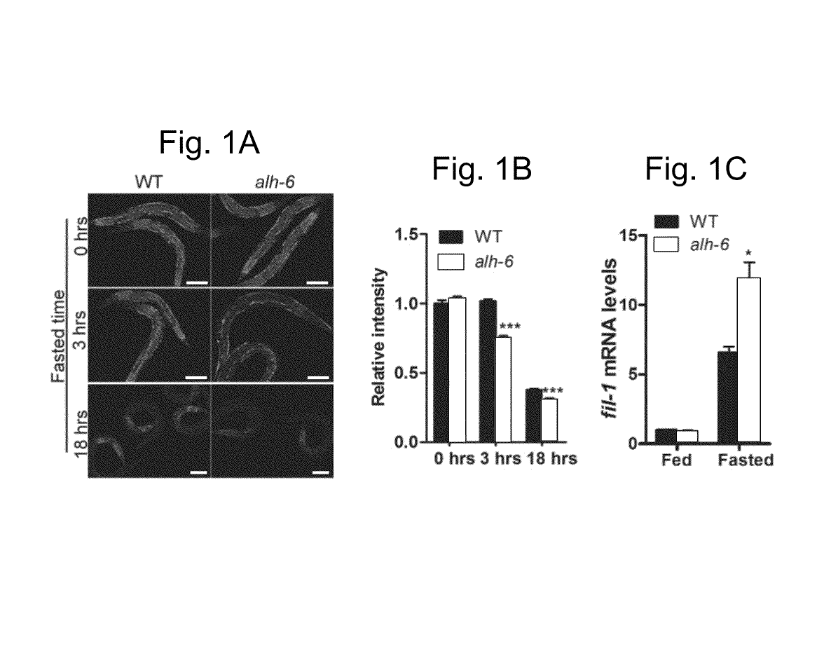

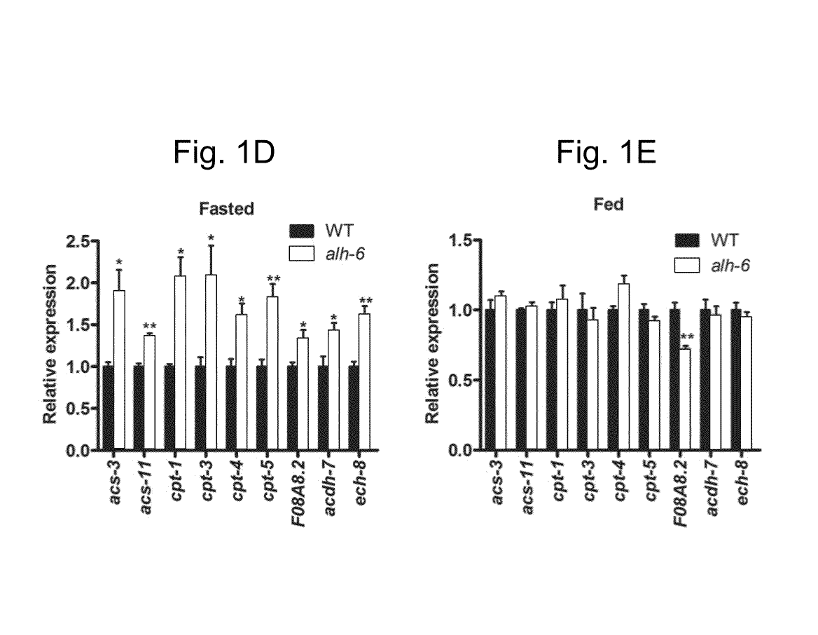

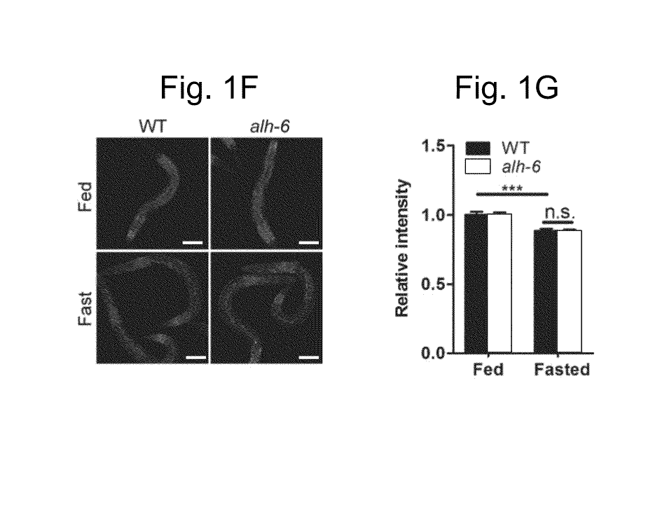

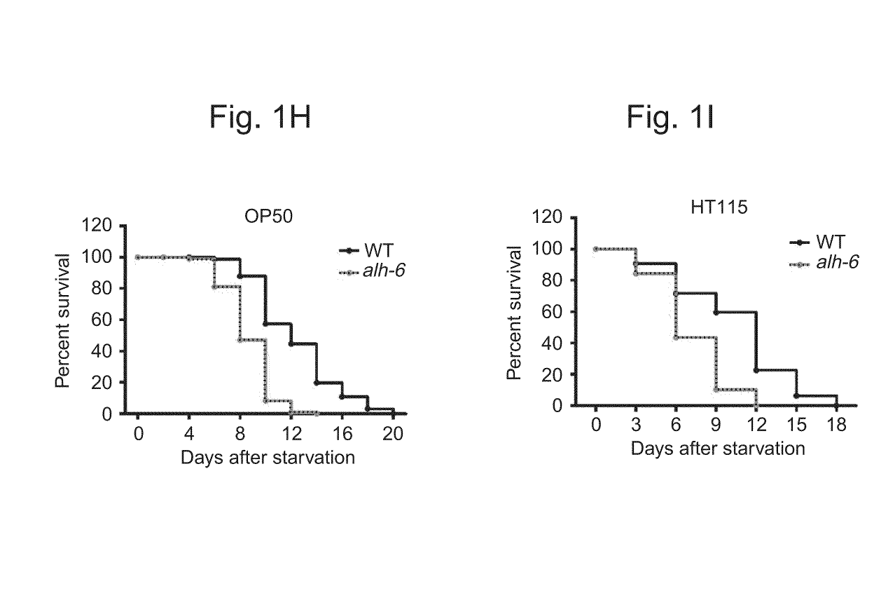

In this study, we asked whether mutation of alh-6 would affect lipid homeostasis. We first compared the fat content between wild-type and alh-6 mutant worms. When fed the standard Escherichia coli OP50 diet ad libitum, wild-type and alh-6 mutant worms store similar levels of intestinal fat as measured by Nile Red staining (FIG. 1a,1b) or Oil Red 0 staining (FIG. 6b). However, within a short 3 h exposure to starvation, alh-6 mutants rapidly mobilized intestinal lipids as compared with wild-type worms, which had yet to measurably use these nutrient stores (FIG. 1a,1b and FIG. 6b). Following a 18 h long-term starvation period, alh-6 mutants continued the hypermobilization of intestinal fat when compared with wild-type animals, which at this time point had also significantly depleted stored lipids (FIG. 1a, 1b). Thus, alh-6 mutants further enhance the mobilization of stored fat in response to food deprivation. This data indicates that alh-6 regulates lipid mobilization during starvation and implies that proline catabolism is coupled with lipid metabolism in response to nutrient depletion.

We next examined the expression of genes involved in lipid metabolism. Under starvation conditions, lipid synthesis is halted via transcriptional repression of fatty acid synthesis enzymes. The expression of fatty acid synthesis enzymes were comparably inhibited in wild type and alh-6 mutant worms in response to fasting, indicating that the enhanced depletion of fat was not regulated at the level of de novo synthesis. The expression levels of pod-2/ACC1 and fasn-1/FASN, the key enzymes in fatty acid synthesis, were comparably inhibited in wild-type and alh-6 mutant worms in response to fasting (FIG. 6c). However, the expression of the fasting-induced lipase-1 (fil-1), a key lipolytic enzyme responsible for the C. elegans starvation response, was significantly induced in alh-6 mutants during starvation above the measured increase in starved wild-type animals (FIG. 1c). Under fasted conditions, fat is used through mitochondrial and peroxisomal FAO. We tested the expression of all annotated FAO enzymes in animals starved for 3 h (Tables 2 and 3). Consistent with the enhanced fat mobilization, alh-6 mutants exhibited increased expression of several FAO enzymes, specifically under fasted (FIG. 1d and Table 3) but not well-fed conditions (FIG. 1e). These enzymes constitute several main steps of mitochondrial and peroxisomal FAO pathways. Thus, fasted alh-6 mutants enhance lipid mobilization characterized by increased expression of genes involved in lipolysis and FAO but not de novo lipogenesis.

TABLE 3
Expression data of all annotated FAO genes in alh-6
mutant worms after three hours of fasting.

| | | Fold change | |
|---|---|---|---|
| Enzymes | Genes | (fasted alh-6/fasted WT) | P value |
| Acyl-CoA | acs-2 | 0.75 | 0.133 |
| Synthetase | acs-3 | 1.90 | 0.026 |
| | acs-11 | 1.37 | 0.002 |
| | acs-13 | 1.07 | 0.623 |
| | acs-18 | 0.89 | 0.353 |
| | R09E10.4 | 0.97 | 0.866 |
| Carnitine | cpt-1 | 2.07 | 0.010 |
| Palmitoyl | cpt-3 | 2.09 | 0.043 |
| Transferase | cpt-4 | 1.62 | 0.020 |
| | cpt-5 | 1.83 | 0.009 |
| | cpt-6 | 1.43 | 0.058 |
| Acyl-CoA | acdh-1 | 0.98 | 0.937 |
| Dehydrogenase | acdh-2 | 0.77 | 0.116 |
| | acdh-3 | 1.12 | 0.445 |
| | acdh-7 | 1.43 | 0.048 |
| | acdh-8 | 0.98 | 0.885 |
| | acdh-9 | 1.29 | 0.099 |
| | acdh-12 | 1.17 | 0.256 |
| Mitochondrial | ech-1 | 1.02 | 0.932 |
| ECH/HACD | ech-2 | 0.99 | 0.933 |
| | ech-4 | 1.09 | 0.759 |
| | ech-5 | 0.78 | 0.332 |
| | ech-6 | 1.00 | 0.976 |
| | ech-7 | 1.01 | 0.863 |
| | hacd-1 | 0.81 | 0.420 |
| | B0272.3 | 1.23 | 0.142 |
| | F54C8.1 | 1.12 | 0.400 |
| | T08B2.7 | 1.17 | 0.140 |
| Mitochondrial | F53A2.7 | 0.90 | 0.495 |
| Thiolase | B0303.3 | 1.18 | 0.316 |
| Acyl-CoA | acox-1 | 1.30 | 0.053 |
| Oxidase | F08A8.2 | 1.34 | 0.036 |
| | F08A8.3 | 1.19 | 0.354 |
| | F08A8.4 | 0.95 | 0.745 |
| | F59F4.1 | 0.99 | 0.912 |
| | C48B4.1 | 0.97 | 0.625 |
| Peroxisomal | ech-3 | 1.36 | 0.064 |
| ECH/HACD | ech-8 | 1.62 | 0.006 |
| | ech-9 | 0.61 | 0.288 |
| Peroxisomal | T02G5.7 | 1.46 | 0.588 |
| Thiolase | | | |

We identified several alh-6-sensitive FAO enzymes that were up-regulated to enhance the wild-type fasting response (FIG. 6d). In addition, we also discovered an increase in the expression of a subset of FAO genes that results in the derepression of targets that are inhibited in starved wild-type animals (FIG. 1e). Some FAO genes were surprisingly inhibited during starvation, indicating they may not be generally essential for the physiologic response to fasting, but rather specifically respond to the fasted state in the context of the alh-6 mutant background, when amino acid metabolism is compromised. While not being bound to any particular theory, C. elegans utilize unique FAO enzymes in response to distinct metabolic stress conditions; some metabolic enzymes can have overlapping functions, can be activated in response to specific cellular needs, and/or become activated in response to the severity of the levels of metabolic homeostatic imbalance. These data indicate that not all FAO enzymes are universally used under all starvation conditions, but rather some may specifically respond to the fasted state in the context of the alh-6 mutant background.

As mutations in alh-6 cause premature ageing in a diet-dependent manner, we asked whether the enhanced lipid mobilization phenotype identified herein was also dependent on diet. Remarkably, the rapid depletion of intestinal lipid stores was abrogated when alh-6 mutants were raised on another common C. elegans diet, the E. coli K-12 strain HT115. Specifically, on this dietary regimen, alh-6 mutants exhibited comparable levels of fat mobilization in response to fasting (FIG. 1f,1g) and showed no significant changes in the expression of FAO genes (FIG. 1f) when compared with wild-type controls. Thus, the diet ingested before starvation establishes an organism's metabolic adaptation program during food deprivation.

We also asked whether mutation of alh-6 affected animal survival during fasting. We found that alh-6 mutants display a significantly reduced animal survival rate in response to starvation (FIG. 1h and Table 4), further indicating that alh-6 is an important regulator of fasting adaptation. Intriguingly, the reduced survival of alh-6 mutant worms during fasting is not dependent on the diet before starvation (FIG. 1i and Table 4), suggesting the role of alh-6 for survival during acute and long-term fasting are different.

TABLE 4
Starvation survival data.
Number of surviving animals¹ (% of day 0)

| | | |
|---|---|---|
| | | alh-6 |
| Days | WT | (lax105) |

OP50:

| | | | | |
|---|---|---|---|---|
| 0 | 157 | (100%) | 206 | (100%) |
| 2 | 159 | (100%) | 212 | (100%) |
| 4 | 160 | (100%) | 204 | (99%) |
| 6 | 155 | (99%) | 167 | (81%) |
| 8 | 138 | (88%) | 97 | (47%) |
| 10 | 90 | (57%) | 17 | (8%) |
| 12 | 70 | (45%) | 2 | (1%) |
| 14 | 31 | (20%) | 0 | (0%) |
| 16 | 17 | (11%) | | |
| 18 | 5 | (3%) | | |
| 20 | 0 | (0%) | | |

HT115:

| | | | | |
|---|---|---|---|---|
| 0 | 141 | (100%) | 108 | (100%) |
| 3 | 128 | (91%) | 91 | (84%) |
| 6 | 101 | (72%) | 47 | (44%) |
| 9 | 84 | (60%) | 11 | (10%) |
| 12 | 32 | (23%) | 0 | (0%) |
| 15 | 9 | (6%) | | |
| 18 | 0 | (0%) | | |

OP50:

| | | | alh-6; | | | | alh-6; | |
|---|---|---|---|---|---|---|---|---|
| Days | skn-1/nTi | | skn-1/nTi | | skn-1 | | skn-1 | |
| 0 | 204 | (100%) | 210 | (100%) | 110 | (100%) | 100 | (100%) |
| 2 | 220 | (100%) | 198 | (94%) | 112 | (100%) | 100 | (100%) |
| 4 | 207 | (100%) | 197 | (94%) | 108 | (98%) | 102 | (100%) |
| 6 | 199 | (98%) | 192 | (91%) | 104 | (95%) | 98 | (98%) |
| 8 | 198 | (97%) | 163 | (78%) | 103 | (94%) | 77 | (77%) |
| 10 | 168 | (82%) | 111 | (53%) | 100 | (91%) | 75 | (75%) |
| 12 | 166 | (81%) | 56 | (27%) | 93 | (85%) | 50 | (50%) |
| 14 | 144 | (71%) | 20 | (10%) | 69 | (63%) | 19 | (19%) |
| 16 | 134 | (66%) | 5 | (2%) | 52 | (47%) | 12 | (12%) |
| 18 | 107 | (52%) | 2 | (1%) | 32 | (29%) | 2 | (2%) |
| 20 | 51 | (25%) | 0 | (0%) | 22 | (20%) | 0 | (0%) |
| 22 | 16 | (8%) | | | 6 | (5%) | | |
| 24 | 0 | (0%) | | | 0 | (0%) | | |

¹Data represent the average from two biological replicates from each condition.

### Example 4

### SKN-1 Mediates Lipid Metabolism Responses in Alh-6 Mutants

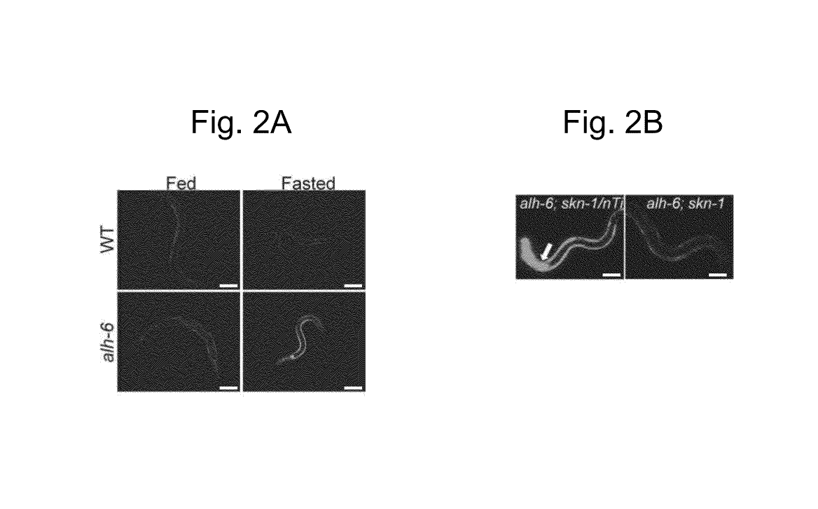

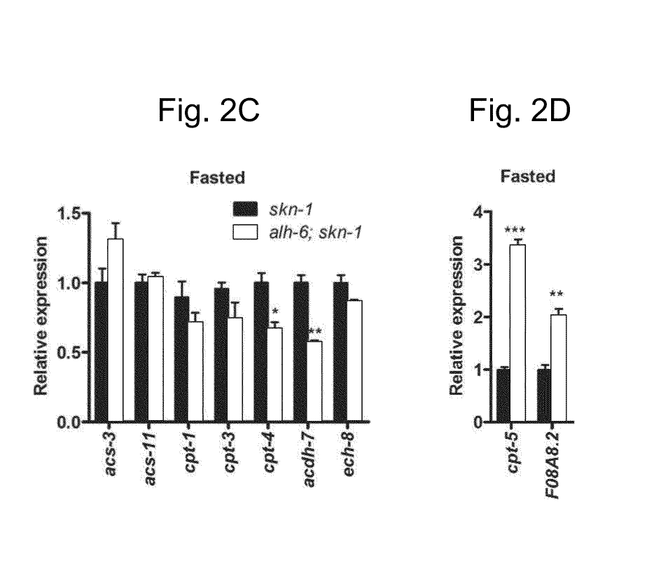

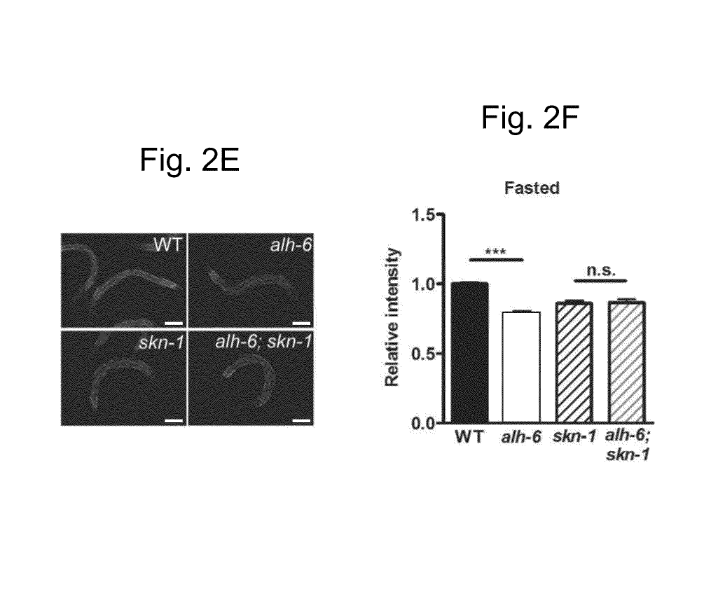

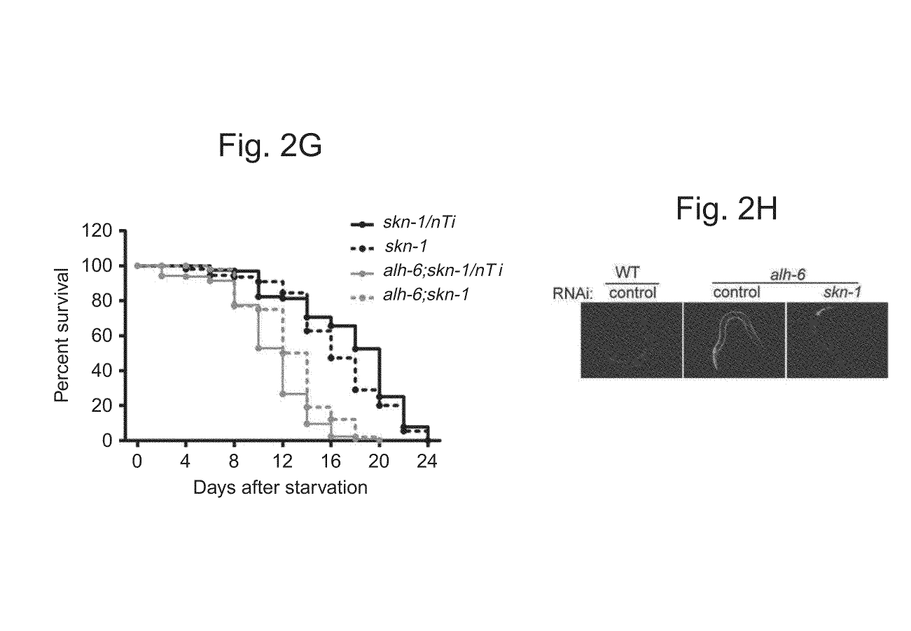

The increased expression of FAO genes in fasted alh-6 mutants indicates the existence of a transcriptional response that monitors and responds to perturbations in cellular proline metabolism. A role for the transcription factor SKN-1 has been documented under conditions of oxidative stress and lifespan extension, where nutrient availability is either perceived as reduced or is actually reduced. Furthermore, we recently found that gain-of-function mutations in skn-1 induce a starvation-like state. As such, we proposed that a SKN-1-mediated transcriptional program could mechanistically link proline and fatty acid metabolism. Under well-fed conditions, the expression of the SKN-1 transcriptional activity reporter gst-4p::GFP was similar between juvenile wild-type and alh-6 mutant worms (FIG. 2a).

However, when starved, the SKN-1 reporter was dramatically activated in alh-6 mutants but not in wild-type controls (FIG. 2a). Furthermore, loss of SKN-1 function through null mutation substantially reduced the fasting-dependent activation of the SKN-1 reporter in alh-6 mutants (FIG. 2b). We conclude that alh-6 mutants activate SKN-1 during food deprivation.

We next asked whether SKN-1 mediated the enhanced mobilization of stored lipids in fasted alh-6 mutants. We found that seven out of nine FAO genes with increased expression in the fasted alh-6 mutants were no longer up-regulated in the absence of SKN-1 (FIG. 2c). Moreover, six of these seven SKN-1 dependent genes contain three to six conserved SKN-1 binding sites in their promoters, indicating that they may be directly regulated by SKN-1. The expression of the carnitine palmitoyl transferase (CPT) gene cpt-5 and the peroxisomal acyl-coenzyme A oxidase gene F08A8.2 were activated independently of SKN-1 in the alh-6 mutants during fasting (FIG. 2d), indicating the existence of other compensatory pathway(s) that function in parallel to SKN-1. Most importantly, a loss-of-function mutation in skn-1 abrogated the enhanced depletion of intestinal lipid stores observed in alh-6 mutant worms after fasting (FIG. 2e,2f), indicating an essential role for SKN-1 in mediating this fasting metabolic response. However, mutation of skn-1 could not significantly reverse the reduced starvation survival rate of alh-6 mutant worms (FIG. 2g); further indicating the mechanistic differences between the lipid metabolism and survival responses in fasted alh-6 mutants.

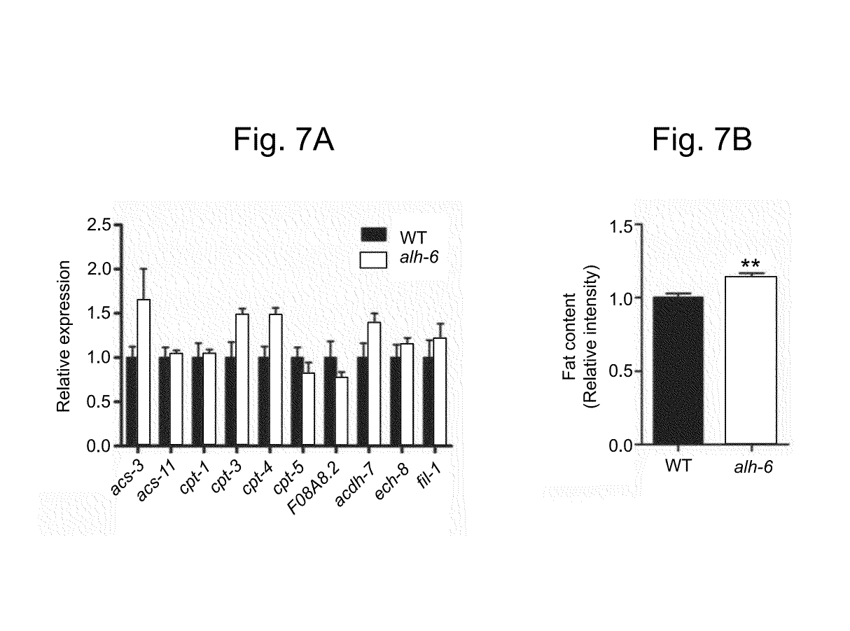

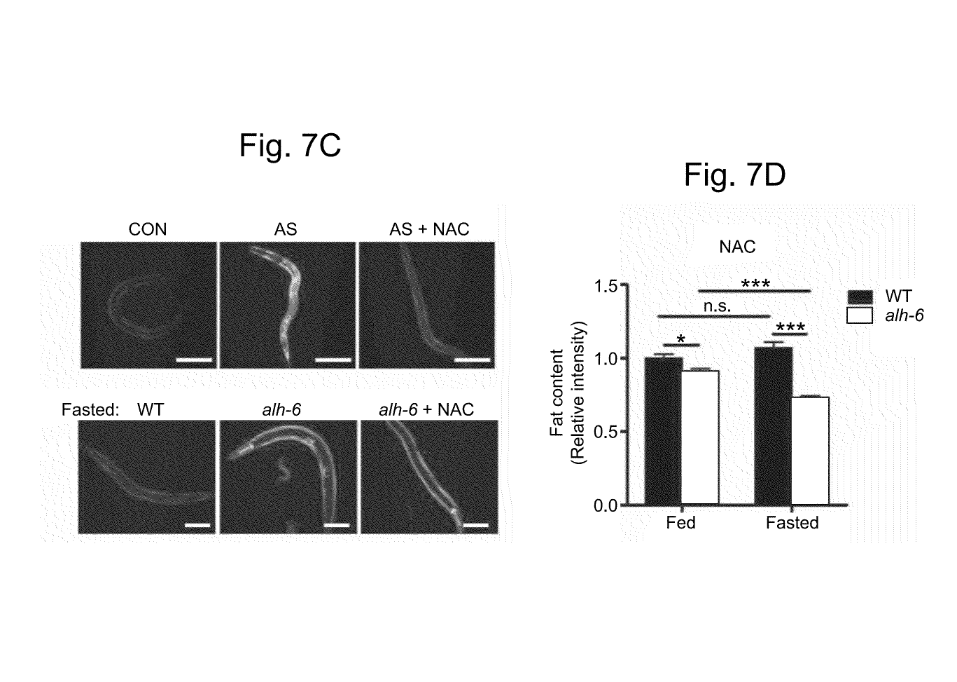

We previously reported that alh-6 mutations were capable of activating the SKN-1 reporter under fed conditions, but only after day 3 of the adult reproductive period. Despite activation of SKN-1 at this time point in adult life, these alh-6 mutants did not induce a similar transcriptional change in FAO genes (FIG. 7a) and do not reduce levels of stored fat (FIG. 7b). These findings indicate a phenotypic difference between the same SKN-1-inducing mutation under different physiologic contexts, which suggests that the SKN-1-mediated lipid response represents a specific metabolic response to the alh-6 mutation during starvation, and not merely an indirect side effect of global SKN-1 activation.

In our previous study, we also reported that accumulation of the alh-6 substrate P5C and the subsequent generation of mitochondrial reactive oxygen species (ROS), such as hydrogen peroxide, are responsible for the premature ageing phenotype observed in adult alh-6 mutants. Treatment with the antioxidant N-acetylcysteine (NAC) completely abrogated the shortened lifespan of alh-6 mutants. As such, we evaluated a role for mitochondrial ROS in alh-6-mediated fasting lipid responses following NAC treatment. We found that although NAC treatment blocked the SKN-1 reporter activation induced by exposure to arsenite, an inducer of oxidative stress, NAC had no effect on the SKN-1 activation observed in fasted alh-6 mutants (FIG. 7c). Moreover, NAC-treated alh-6 mutant worms still exhibited accelerated fat mobilization in response to fasting (FIG. 7d). While not being bound to any particular theory, these data suggest, that mitochondrial oxidative stress is not involved in SKN-1 activation and the enhanced lipid metabolism observed in fasted alh-6 worms. As SKN-1 can respond to multiple types of cellular stress, it is possible that additional, non-oxidative stress signals caused by P5C accumulation are responsible for the observed SKN-1 activation and lipid changes.

### Example 5

### SKN-1 Protects Against Diet-Induced Fat Accumulation

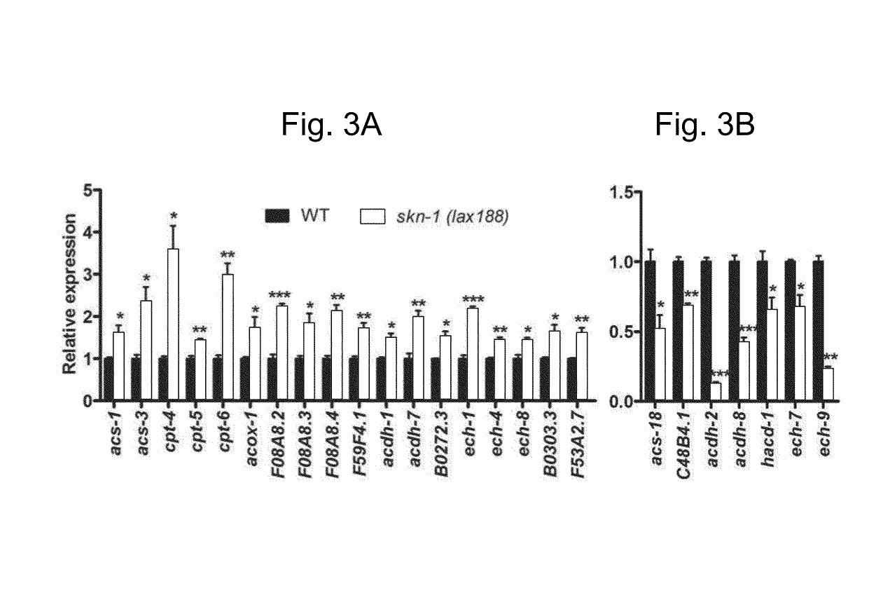

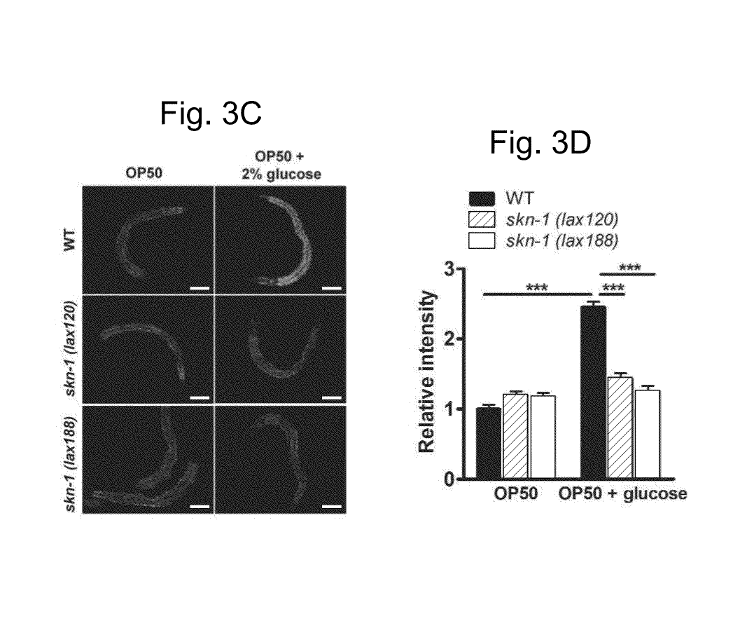

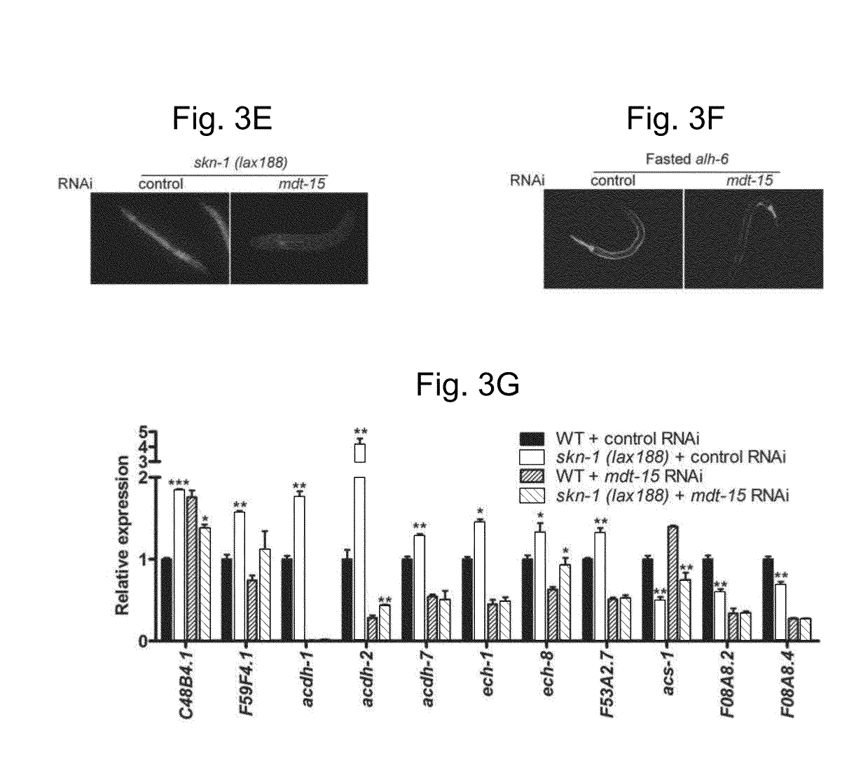

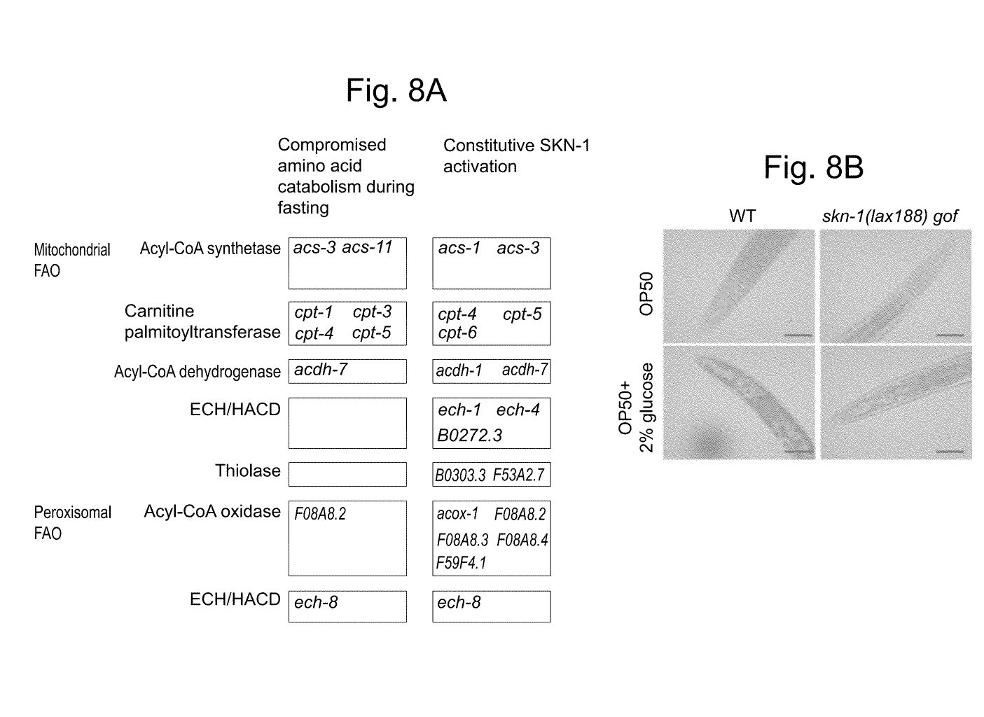

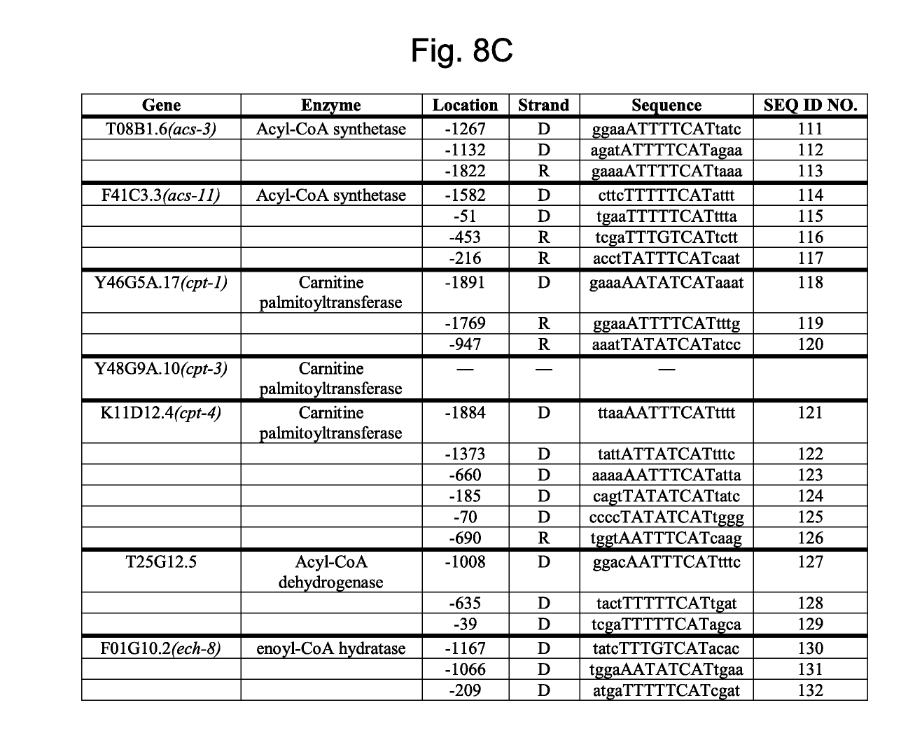

Subsequently, we tested whether skn-1 gain-of-function mutations could induce a similar transcriptional response, and more importantly, if they result in a change in stored lipids. We discovered that well-fed skn-1 gain-of-function mutant worms up-regulated a large number of FAO genes (FIG. 3a), 83% (15/18) of which contain at least one SKN-1 binding site in their promoters, which is consistent with our previous observation that ad libitum-fed skn-1 gain-of-function animals behave as if they are starved. Intriguingly, some FAO genes were found to be down-regulated in skn-1 gain-of-function mutants as compared with wild-type controls (FIG. 3b), further supporting the idea that unique FAO enzymes are differentially mobilized in response to particular metabolic stresses. Although, there was a larger set of lipid metabolism genes altered in the skn-1 gain-of-function mutants, there was a clear overlap with the genes increased in the alh-6 mutants during fasting (FIG. 8a). This gene expression pattern indicates a SKN-1-dependent pathway for inducing an organism-level metabolic response that is defined by the activation of fatty acid utilization pathways in both the skn-1 gain-of-function mutants and SKN-1-activating alh-6 mutants under conditions of fasting. We then measured the fat content of those gain-of-function mutant worms. Although transcriptionally poised for increased oxidation of stored fat, well-fed skn-1 gain-of-function mutant animals exhibited relatively similar levels of fat content compared with well-fed wild-type controls as measured by Nile Red staining (FIG. 3c,3d), and a minor decrease of fat as revealed by Oil Red O staining (FIG. 8b). We hypothesized that the induction of FAO enzymatic activity in mutants with constitutive SKN-1 activation might only significantly impact lipid homeostasis, at the organismal level, under conditions of metabolic stress. We thus examined the function of constitutively activated SKN-1 in animals fed a high carbohydrate diet (HCD), which serves as model that mimics the diet-induced obesity observed in mammals. We found that addition of 2% glucose to the standard diet could significantly induce a 250% increase in stored intestinal fat in wild-type C. elegans, as compared with worms feeding on a normal diet (FIG. 3c, 3d and FIG. 8b). Strikingly, when skn-1 gain-of-function mutants were fed the HCD, they did not manifest this increased lipid phenotype (FIG. 3c, 3d and FIG. 8b). These data suggest that constitutive SKN-1 activation can transcriptionally predispose animals to successfully cope with dietary insults, and that this adaptive capacity is capable of suppressing the lipid accumulation phenotype resulting from a HCD.

### Example 6

### Aldh4a1 and Nrf2 Regulate FAO Genes in Human Cells

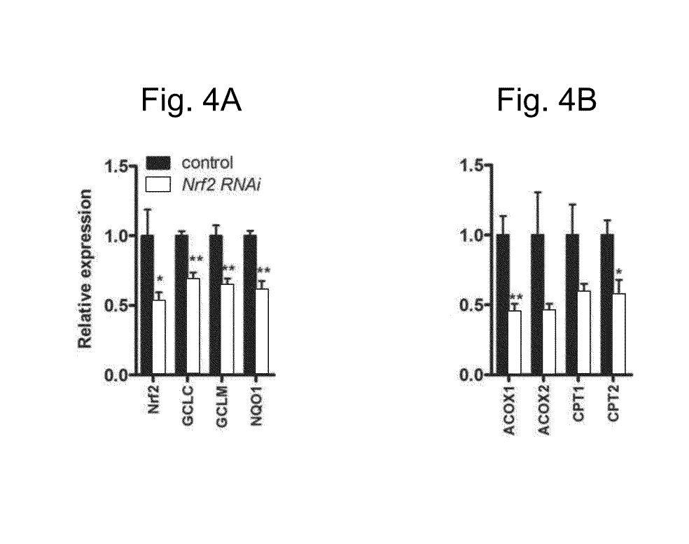

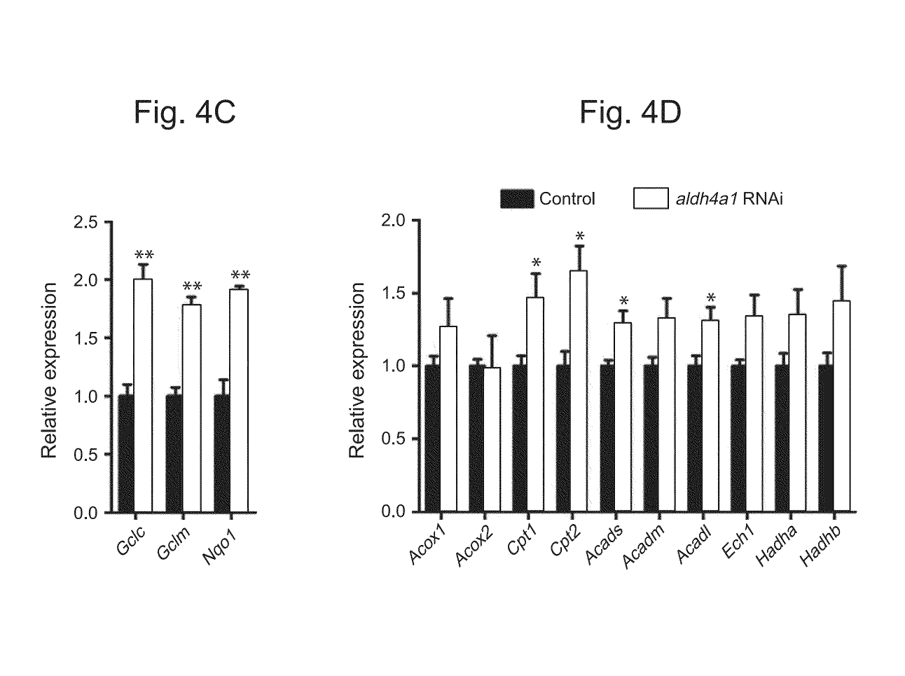

We next examined the possible conservation of the alh-6/skn-1 pathway in human cells. We first asked whether Nrf2, the human orthologue of SKN-1, also regulated the expression of FAO genes in human cells. Although Nrf2 activity has been linked to cancer cell metabolism and lipid biosynthesis in rodents, its role in regulating FAO has not been established. We found that RNA interference (RNAi)-mediated knockdown of Nrf2 inhibited the expression of canonical Nrf2 target genes (FIG. 4a) and also several FAO genes in 293T cells (FIG. 4b), indicating that Nrf2 is a regulator of FAO genes in human cells. Next, we performed small interfering RNA (siRNA) knockdown of aldh4a1, the human orthologue of worm alh-6, and examined the effects on gene expression. Remarkably, aldh4a1 RNAi not only induced the expression of Nrf2 targets (FIG. 4c), which is indicative of Nrf2 activation, but also induced the expression of a subset of FAO genes (FIG. 4d). These data implicate that the SKN-1/Nrf2-mediated regulatory axis between proline and lipid metabolism has functional conservation from invertebrates to humans.

### Example 7

### MDT-15 Co-Regulates Lipid Metabolism with SKN-1

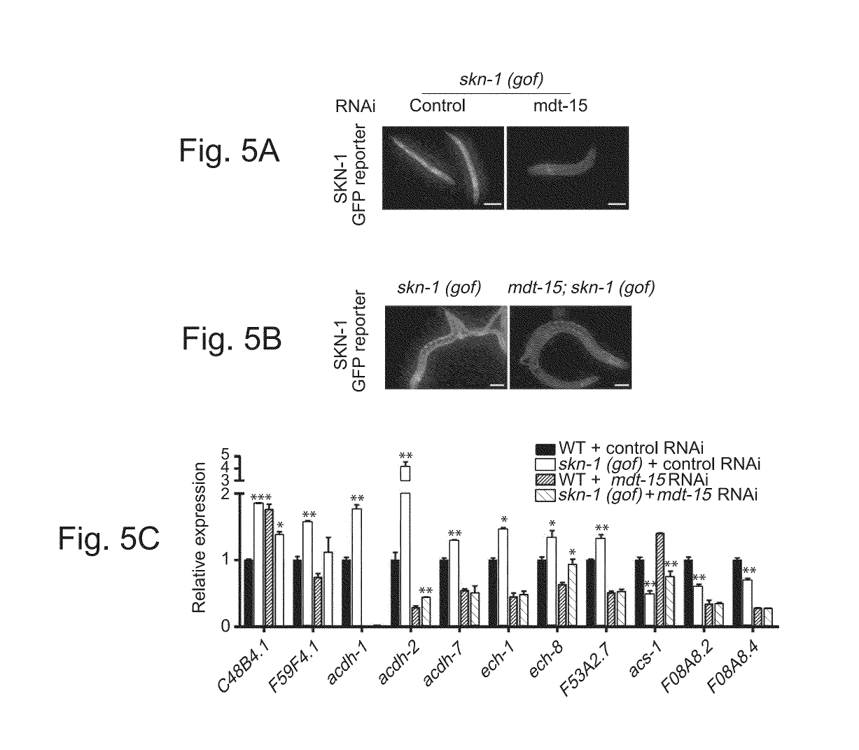

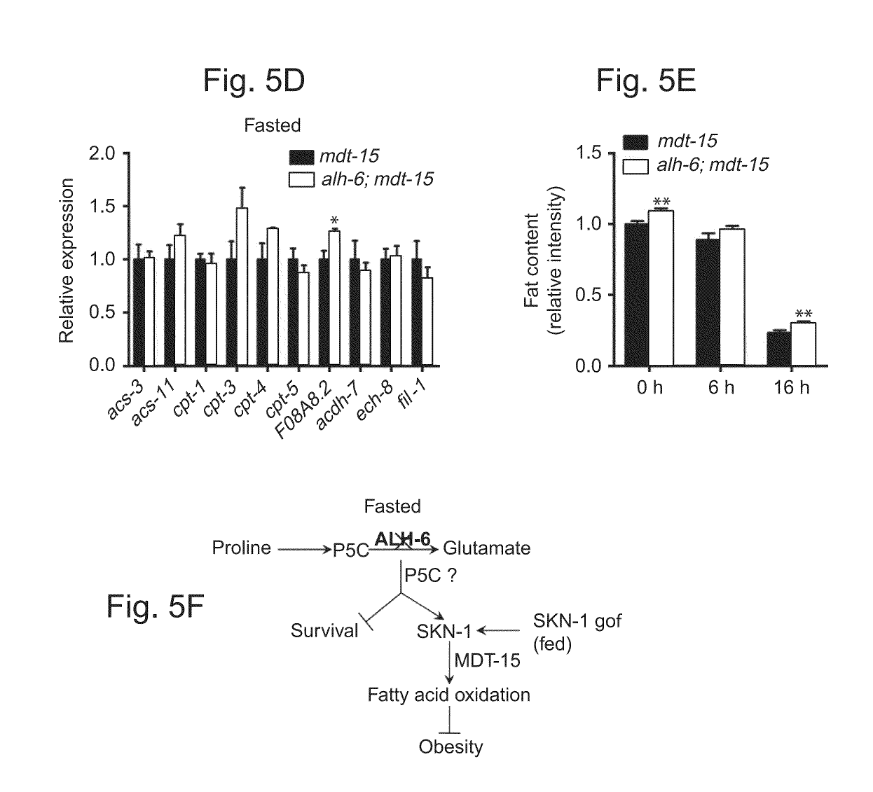

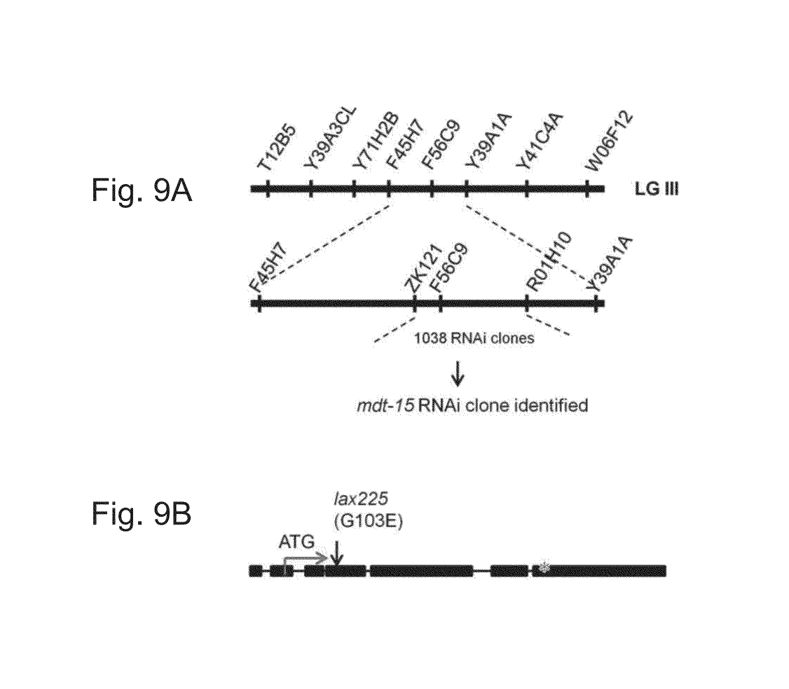

In light of the multitude of responses that are influenced by SKN-1/Nrf2, we predicted that the SKN-1/Nrf2 lipid metabolism response we identified would require additional transcriptional co-regulators. To identify possible co-regulators of SKN-1 in modulating lipid metabolism, we first screened an RNAi library targeting all annotated transcriptional regulators and DNA-binding proteins in C. elegans, looking for suppression of the SKN-1 reporter activation observed in the skn-1 gain-of-function mutants. We discovered that mdt-15 was required for SKN-1 reporter activation, as RNAi targeting mdt-15 significantly abolished the reporter activation (FIG. 5a). Moreover, in a complementary approach, we performed a classical ethyl methanesulfonate (EMS) mutagenesis screen for suppressors of the SKN-1 reporter activation in the skn-1 gain-of-function mutant background. We isolated a single complementation group that mapped to the center of LGIII and identified a Gly to Glu mutation in MDT-15 (FIG. 5b and FIG. 9a, 9b). MDT-15 is a transcriptional regulator of lipid metabolism and has been found to physically interact with SKN-1. Similarly, the activation of the SKN-1 reporter in fasted alh-6 mutants also required mdt-15 (FIG. 3f), whereas nhr-49, another critical lipid transcriptional regulator, is dispensable for such activation. We then subsequently tested the role for MDT-15 in SKN-1-mediated lipid metabolism by examining the effect of mdt-15 RNAi on lipid gene expression in the skn-1 gain-of-function mutants. These mutants also display enhanced expression of FAO genes when raised on the control RNAi bacteria HT115 (FIG. 5c). However, it is notable that the gene expression changes observed are not identical to those when animals were fed the OP50 E. coli B diet (FIG. 3a,3b), further supporting the diet-dependent response of SKN-1 function in lipid metabolism. RNAi knockdown of mdt-15 largely abolished the effects of skn-1 gain-of-function mutation on FAO gene expression (FIG. 5c), suggesting MDT-15 is a critical cofactor for the transcription of these targets. Moreover, in the mdt-15 mutant background, alh-6 mutant animals no longer exhibited the increased expression of FAO genes (FIG. 5d) or enhanced fat mobilization in response to fasting (FIG. 5e). Together, our results refine the molecular mechanisms by which SKN-1 and MDT-15 cooperate to maintain lipid homeostasis and define MDT-15 as a co-regulator of SKN-1-dependent lipid metabolism.

Molecular mechanisms that link glucose and lipid metabolic pathways have been previously documented. In this study, we reveal a functional link between amino and fatty acid metabolism, and identify SKN-1 as a transcriptional switch that coordinates the utilization of these two critical nutrients. During food deprivation, fatty acids and amino acids comprise the major cellular energy resources. However, if amino acid metabolism is compromised, organisms respond to this defect by switching on a SKN-1 and MDT-15 mediated transcription program that further activates FAO. We find that constitutively activated SKN-1 can significantly protect animals against the increased lipid storage phenotype when fed a HCD. This finding implicates SKN-1/Nrf2 as a potential target for the treatment of obesity and related metabolic diseases.

### Example 8

### Discussion

In this study, we reveal a novel link between proline and lipid metabolism, and identify a SKN-1/Nrf2-dependent mechanism that coordinates these two metabolic pathways (FIG. 5f). How does mutation of alh-6 lead to SKN-1 activation and lipid responses during fasting? A possible mechanism is that accumulation of the alh-6 substrate P5C induces SKN-1 activation and fat mobilization. A recent study in mammalian adipose cells has reported that during nutrient withdrawal, the activation of prodh, the enzyme producing P5C, can induce lipase expression. This finding supports the model for P5C as a conserved metabolic signal in activating SKN-1 and regulating fat mobilization during starvation. Generation and utilization of animals with mutations in or reduced expression of prodh, the P5C generating enzyme, will be valuable for testing this theory. Although Barbato et al. (Barbato et al., Cell Death Differ., 21, 113-123, 2014) identified a role for ROS, this could represent the differences between our experimental models and readouts: apoptosis and inflammation in 3T3 cells versus organismal lipid depletion, or the inherent differences in responses for specific tissues. A more thorough understanding of the coordination of such responses will be of significant interest for future studies.

SKN-1 is an essential transcription factor mediating cellular stress responses, such as oxidative stress and immune defense. Recent gene profile analyses indicate that SKN-1 may also be an important regulator of metabolism. In this study, we identify a physiological role for SKN-1 in metabolism, coordinating proline catabolism with lipid utilization during fasting. SKN-1 is thus a critical transcription factor that responds to diverse cellular stresses, including metabolic stress. Disruption of alh-6-dependent proline catabolism during fasting induces changes in the expression of several FAO genes, most of which are regulated in a SKN-1-dependent manner. Intriguingly, six of the seven SKN-1-dependent genes we identified contain three to six conserved SKN-1-binding sites in their 2 kb promoters (FIG. 8c); a SKN-1-binding site is generally found by chance only once in the same length of the genome. This data indicates that some of these FAO genes may be direct targets of SKN-1.

We find that a subset of FAO genes, whose expression is inhibited by starvation in wild-type animals, is derepressed in fasted alh-6 mutant worms. This finding indicates physiological differences of the collection of FAO genes in the genome. We propose that C. elegans use unique FAO enzymes in response to distinct metabolic stress conditions: some metabolic enzymes can have overlapping functions and/or can be activated in response to specific cellular needs.

Another interesting finding of our study is that compromised alh-6-mediated proline catabolism regulates lipid metabolism during fasting in a diet-dependent manner. Although the response is triggered under a condition without food, our data suggests that dietary intake before food deprivation could predetermine an organism's response during starvation. The different nutritional composition between the OP50 and HT115 diets may lead to preferential use of specific energy substrates. We propose that, when fasted, animals previously fed an OP50 diet may rely more on mitochondrial alh-6 proline catabolism than those fed the HT115 diet. When alh-6 is mutated, animals fed the OP50 diet might be more stressed when exogenous nutrients are no longer available. This condition thereby activates the lipid metabolism response through SKN-1 and MDT-15. Intriguingly, the diet consumed before fasting can have significant effects on mouse behavior during food deprivation, which suggests that dietary pre-determination of the adaptive response to starvation is also evolutionarily conserved.

Abnormal fat accumulation induced by diet underlies multiple metabolic diseases, such as obesity and type II diabetes. We and others show that a diet supplemented with high glucose can induce massive lipid accumulation in C. elegans, indicating the possibility of using this as a model to study diet-induced fat accumulation. In this study, we find that skn-1 gain-of-function mutation protects animals against the increased lipid storage phenotype when fed a HCD. This finding implicates SKN-1 as a potential target for the treatment of abnormal lipid metabolism. Furthermore, Nrf2 can similarly regulate FAO genes in human cells revealing the evolutionary importance of this cellular metabolic response system. Thus, studies regarding the possible use of Nrf2 pathway agonists for regulating lipid metabolism and improving its related metabolic diseases will be of high clinical importance.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method for treating obesity in a subject in need thereof, the method comprising: administering to the subject an agent capable of increasing Nrf2/SKN-1 activity, MDT-15 activity, or a combination thereof in the subject.

2. The method of claim 1, wherein the agent enhances utilization of stored lipids in the subject.

3. The method of claim 1, further comprising altering the expression of a gene selected from the group consisting of: fil-1, a fatty acid oxidation enzyme, and combinations thereof.

4. The method of claim 3, wherein the fatty acid oxidation enzyme is selected from the group consisting of acs-3, acs-11, cpt-1, cpt-3, cpt-4, cpt-5, F08A8.2, acdh-7, ech-8, GCLC, GCLM, NQO1, ACOX1, ACOX2, CPT1, CPT2, and combinations thereof.

5. The method of claim 1, wherein the subject is predisposed to obesity.

6. A method for protecting a subject in need thereof from obesity, the method comprising increasing Nrf2/SKN-1 activity, MDT-15 activity, or a combination thereof in the subject.

7. The method of claim 6, further comprising altering the expression of a gene selected from the group consisting of: fil-1, a fatty acid oxidation enzyme, and combinations thereof.

8. The method of claim 7, wherein the fatty acid oxidation enzyme is selected from the group consisting of acs-3, acs-11, cpt-1, cpt-3, cpt-4, cpt-5, F08A8.2, acdh-7, ech-8, GCLC, GCLM, NQO1, ACOX1, ACOX2, CPT1, CPT2, and combinations thereof.

9. The method of claim 6, wherein the subject is predisposed to obesity.

10. A method for monitoring an efficacy of a treatment for obesity in a subject, the method comprising:
(a) measuring a first level of Nrf2/SKN-1 in a first sample obtained from the subject before the onset of the treatment;
(b) measuring a second level of Nrf2/SKN-1 in a second sample obtained from the subject after the onset of treatment;
(c) comparing the measured second level of Nrf2/SKN-1 to the measured first level of Nrf2/SKN-1; and
(d) determining that the treatment is effective if the second level of Nrf2/SKN-1 is greater than the first level of Nrf2/SKN-1 or that the treatment is not effective if the second level of Nrf2/SKN-1 is equal or less than the first level of Nrf2/SKN-1.

11. The method of claim 10, wherein the treatment is a fasting diet.

12. The method of claim 10, further comprising altering the expression of a gene selected from the group consisting of fil-1, a fatty acid oxidation enzyme, and combinations thereof.

13. The method of claim 12, wherein the fatty acid oxidation enzyme is selected from the group consisting of acs-3, acs-11, cpt-1, cpt-3, cpt-4, cpt-5, F08A8.2, acdh-7, ech-8, GCLC, GCLM, NQO1, ACOX1, ACOX2, CPT1, CPT2, and combinations thereof.


SEQUENCE LISTING (132 sequences)

SEQ ID NO: 1 - DNA, 21 bp, Artificial Sequence
  synthetic construct
aaggagatgagaatgactgat

SEQ ID NO: 2 - DNA, 18 bp, Artificial Sequence
  synthetic construct
gttccgacatggtgacta

SEQ ID NO: 3 - DNA, 18 bp, Artificial Sequence
  synthetic construct
cacgattcaagcaacttc

SEQ ID NO: 4 - DNA, 22 bp, Artificial Sequence
  synthetic construct
ttcacttccttattctccatta

SEQ ID NO: 5 - DNA, 22 bp, Artificial Sequence
  synthetic construct
gcttattggaattatgaagaag

SEQ ID NO: 6 - DNA, 18 bp, Artificial Sequence
  synthetic construct
ggaccttagtgatgtgat

SEQ ID NO: 7 - DNA, 18 bp, Artificial Sequence
  synthetic construct
atcaggcagagatcaaga

SEQ ID NO: 8 - DNA, 18 bp, Artificial Sequence
  synthetic construct
ggttccatcacaacaagt

SEQ ID NO: 9 - DNA, 18 bp, Artificial Sequence
  synthetic construct
gcttacaatgtcctatcg

SEQ ID NO: 10 - DNA, 20 bp, Artificial Sequence
  synthetic construct
tccatcttcttgaataatcg

SEQ ID NO: 11 - DNA, 18 bp, Artificial Sequence
  synthetic construct
attgatactggcgatgaa

SEQ ID NO: 12 - DNA, 20 bp, Artificial Sequence
  synthetic construct
agagattggttatgtaatgc

SEQ ID NO: 13 - DNA, 18 bp, Artificial Sequence
  synthetic construct
tctattgtcgtggagtct

SEQ ID NO: 14 - DNA, 18 bp, Artificial Sequence
  synthetic construct
ggattgcgtcgtattgta

SEQ ID NO: 15 - DNA, 19 bp, Artificial Sequence
  synthetic construct
tacacggattctatgaagt

SEQ ID NO: 16 - DNA, 19 bp, Artificial Sequence
  synthetic construct
tagttgtctgtgattaggt

SEQ ID NO: 17 - DNA, 18 bp, Artificial Sequence
  synthetic construct
ccagcacttcaggatact

SEQ ID NO: 18 - DNA, 19 bp, Artificial Sequence
  synthetic construct
agcagttggtcatagtctt

SEQ ID NO: 19 - DNA, 19 bp, Artificial Sequence
  synthetic construct
gacagcacaattcgtagta

SEQ ID NO: 20 - DNA, 18 bp, Artificial Sequence
  synthetic construct
tctccagccaacatatct

SEQ ID NO: 21 - DNA, 19 bp, Artificial Sequence
  synthetic construct
cacttctacttctcataca

SEQ ID NO: 22 - DNA, 18 bp, Artificial Sequence
  synthetic construct
tcacaagattcaaggatt

SEQ ID NO: 23 - DNA, 18 bp, Artificial Sequence
  synthetic construct
ctctgttctgatagtctt

SEQ ID NO: 24 - DNA, 18 bp, Artificial Sequence
  synthetic construct
cctctcctgaattagtaa

SEQ ID NO: 25 - DNA, 18 bp, Artificial Sequence
  synthetic construct
caggaaccattgctcaag

SEQ ID NO: 26 - DNA, 22 bp, Artificial Sequence
  synthetic construct
attcagaacttcaatagcgtat

SEQ ID NO: 27 - DNA, 18 bp, Artificial Sequence
  synthetic construct
cacaagtcgtcaattctg

SEQ ID NO: 28 - DNA, 18 bp, Artificial Sequence
  synthetic construct
tgagccaatcctaacatt

SEQ ID NO: 29 - DNA, 18 bp, Artificial Sequence
  synthetic construct
accaagtgtgagaagaag

SEQ ID NO: 30 - DNA, 18 bp, Artificial Sequence
  synthetic construct
cgaagaaccagttagcat

SEQ ID NO: 31 - DNA, 19 bp, Artificial Sequence
  synthetic construct
aatcatcaaggagttcaat

SEQ ID NO: 32 - DNA, 18 bp, Artificial Sequence
  synthetic construct
aagagacaccataagagt

SEQ ID NO: 33 - DNA, 21 bp, Artificial Sequence
  synthetic construct
ttctgtcttatgattgaggat

SEQ ID NO: 34 - DNA, 18 bp, Artificial Sequence
  synthetic construct
cttgttggttgtgagttc

SEQ ID NO: 35 - DNA, 19 bp, Artificial Sequence
  synthetic construct
aactcttggaatggtgatg

SEQ ID NO: 36 - DNA, 18 bp, Artificial Sequence
  synthetic construct
aactgctcttggaatgtc

SEQ ID NO: 37 - DNA, 21 bp, Artificial Sequence
  synthetic construct
cgaatgtaactatcaataagg

SEQ ID NO: 38 - DNA, 18 bp, Artificial Sequence
  synthetic construct
atggcggaataatcaatt

SEQ ID NO: 39 - DNA, 18 bp, Artificial Sequence
  synthetic construct
gagttgaaggctattgac

SEQ ID NO: 40 - DNA, 18 bp, Artificial Sequence
  synthetic construct
atattgcggatgaagttc

SEQ ID NO: 41 - DNA, 18 bp, Artificial Sequence
  synthetic construct
aacgcattgacattggaa

SEQ ID NO: 42 - DNA, 19 bp, Artificial Sequence
  synthetic construct
cattggcagtaatcacagt

SEQ ID NO: 43 - DNA, 19 bp, Artificial Sequence
  synthetic construct
gtgtggtgattctcaactc

SEQ ID NO: 44 - DNA, 18 bp, Artificial Sequence
  synthetic construct
ctgctggctcatagtctt

SEQ ID NO: 45 - DNA, 19 bp, Artificial Sequence
  synthetic construct
tggatactgataagtctgt

SEQ ID NO: 46 - DNA, 18 bp, Artificial Sequence
  synthetic construct
ctcgttattggtcatctc

SEQ ID NO: 47 - DNA, 18 bp, Artificial Sequence
  synthetic construct
tgttctgttggctgatag

SEQ ID NO: 48 - DNA, 18 bp, Artificial Sequence
  synthetic construct
tttaggctggtttggtag

SEQ ID NO: 49 - DNA, 18 bp, Artificial Sequence
  synthetic construct
gcttgtagaagttgtatc

SEQ ID NO: 50 - DNA, 18 bp, Artificial Sequence
  synthetic construct
aatgaatccaggagtatc

SEQ ID NO: 51 - DNA, 20 bp, Artificial Sequence
  synthetic construct
tgtggatagcaatcaatctg

SEQ ID NO: 52 - DNA, 19 bp, Artificial Sequence
  synthetic construct
cttcttcttggcaactctc

SEQ ID NO: 53 - DNA, 20 bp, Artificial Sequence
  synthetic construct
gatagtcctggattcattgt

SEQ ID NO: 54 - DNA, 18 bp, Artificial Sequence
  synthetic construct
gcatctcctcgttcatac

SEQ ID NO: 55 - DNA, 18 bp, Artificial Sequence
  synthetic construct
tgctcaggaacttgctaa

SEQ ID NO: 56 - DNA, 19 bp, Artificial Sequence
  synthetic construct
gtcataactgcttgcgtag

SEQ ID NO: 57 - DNA, 18 bp, Artificial Sequence
  synthetic construct
aagttaccagacaagaag

SEQ ID NO: 58 - DNA, 18 bp, Artificial Sequence
  synthetic construct
atagtgattccaacgatt

SEQ ID NO: 59 - DNA, 18 bp, Artificial Sequence
  synthetic construct
gaagagaacaagacgaat

SEQ ID NO: 60 - DNA, 20 bp, Artificial Sequence
  synthetic construct
taagaatactggaacaacat

SEQ ID NO: 61 - DNA, 18 bp, Artificial Sequence
  synthetic construct
cttcaacaactaccgtat

SEQ ID NO: 62 - DNA, 19 bp, Artificial Sequence
  synthetic construct
agcatataacttctcacat

SEQ ID NO: 63 - DNA, 18 bp, Artificial Sequence
  synthetic construct
atctaaccagcctgaatg

SEQ ID NO: 64 - DNA, 18 bp, Artificial Sequence
  synthetic construct
atgccacttctcttgatt

SEQ ID NO: 65 - DNA, 18 bp, Artificial Sequence
  synthetic construct
acgatacaatgttcatac

SEQ ID NO: 66 - DNA, 19 bp, Artificial Sequence
  synthetic construct
aataatctccttccttcta

SEQ ID NO: 67 - DNA, 18 bp, Artificial Sequence
  synthetic construct
cagcagcatcgtctattc

SEQ ID NO: 68 - DNA, 20 bp, Artificial Sequence
  synthetic construct
gttcaataccttctccagtt

SEQ ID NO: 69 - DNA, 18 bp, Artificial Sequence
  synthetic construct
actcactatccactcaag

SEQ ID NO: 70 - DNA, 18 bp, Artificial Sequence
  synthetic construct
cgtatctcatcagcaatg

SEQ ID NO: 71 - DNA, 19 bp, Artificial Sequence
  synthetic construct
ctctgatgttcttgctgat

SEQ ID NO: 72 - DNA, 18 bp, Artificial Sequence
  synthetic construct
atgcttgtcttgcttgat

SEQ ID NO: 73 - DNA, 18 bp, Artificial Sequence
  synthetic construct
gagcactggatatgatac

SEQ ID NO: 74 - DNA, 18 bp, Artificial Sequence
  synthetic construct
ccgagattctattgacaa

SEQ ID NO: 75 - DNA, 21 bp, Artificial Sequence
  synthetic construct
gaggaaggaatcatccatcat

SEQ ID NO: 76 - DNA, 18 bp, Artificial Sequence
  synthetic construct
gaccaccagtagcaacag

SEQ ID NO: 77 - DNA, 21 bp, Artificial Sequence
  synthetic construct
gtattcaacaggcttcttcat

SEQ ID NO: 78 - DNA, 19 bp, Artificial Sequence
  synthetic construct
ccattcatatcggcaacag

SEQ ID NO: 79 - DNA, 18 bp, Artificial Sequence
  synthetic construct
actattcttgttgttgga

SEQ ID NO: 80 - DNA, 18 bp, Artificial Sequence
  synthetic construct
gcatcttgttcttctcta

SEQ ID NO: 81 - DNA, 19 bp, Artificial Sequence
  synthetic construct
gtgtcaactattcctcatt

SEQ ID NO: 82 - DNA, 20 bp, Artificial Sequence
  synthetic construct
ggttccgtattcaataagat

SEQ ID NO: 83 - DNA, 19 bp, Artificial Sequence
  synthetic construct
aataagcagtctcttgatg

SEQ ID NO: 84 - DNA, 19 bp, Artificial Sequence
  synthetic construct
cacttctgtatccttcttc

SEQ ID NO: 85 - DNA, 20 bp, Artificial Sequence
  synthetic construct
tctacttcttaggtgaggaa

SEQ ID NO: 86 - DNA, 20 bp, Artificial Sequence
  synthetic construct
cgtgattggaatctgataac

SEQ ID NO: 87 - DNA, 18 bp, Artificial Sequence
  synthetic construct
gcagccagattagtagaa

SEQ ID NO: 88 - DNA, 18 bp, Artificial Sequence
  synthetic construct
aacaaggtcaacagaagt

SEQ ID NO: 89 - DNA, 18 bp, Artificial Sequence
  synthetic construct
cacttggctgttatgatg

SEQ ID NO: 90 - DNA, 18 bp, Artificial Sequence
  synthetic construct
gttctcctgagtattggt

SEQ ID NO: 91 - DNA, 18 bp, Artificial Sequence
  synthetic construct
gattgtgctgtgaactac

SEQ ID NO: 92 - DNA, 18 bp, Artificial Sequence
  synthetic construct
caacttgaactggatgac

SEQ ID NO: 93 - DNA, 18 bp, Artificial Sequence
  synthetic construct
ctggtgctgttggattag

SEQ ID NO: 94 - DNA, 19 bp, Artificial Sequence
  synthetic construct
atattgcttggtgctctac

SEQ ID NO: 95 - DNA, 22 bp, Artificial Sequence
  synthetic construct
tagtattcattcaggtattgtc

SEQ ID NO: 96 - DNA, 18 bp, Artificial Sequence
  synthetic construct
gctctgtcattgctattg

SEQ ID NO: 97 - DNA, 18 bp, Artificial Sequence
  synthetic construct
tagagtgcttcaacaaga

SEQ ID NO: 98 - DNA, 18 bp, Artificial Sequence
  synthetic construct
atgtccatcaggtcaata

SEQ ID NO: 99 - DNA, 18 bp, Artificial Sequence
  synthetic construct
ccgtccttatctcatcaa

SEQ ID NO: 100 - DNA, 19 bp, Artificial Sequence
  synthetic construct
aactattctctgtgcttct

SEQ ID NO: 101 - DNA, 19 bp, Artificial Sequence
  synthetic construct
acactcacactaggcaatg

SEQ ID NO: 102 - DNA, 18 bp, Artificial Sequence
  synthetic construct
aacactggcaaggcttaa

SEQ ID NO: 103 - DNA, 19 bp, Artificial Sequence
  synthetic construct
acacggtccacagctcatc

SEQ ID NO: 104 - DNA, 22 bp, Artificial Sequence
  synthetic construct
ttgacatactttggaggcaaga

SEQ ID NO: 105 - DNA, 20 bp, Artificial Sequence
  synthetic construct
caaggacgttctcaagtggg

SEQ ID NO: 106 - DNA, 20 bp, Artificial Sequence
  synthetic construct
agagaagggggaaaggacaa

SEQ ID NO: 107 - DNA, 20 bp, Artificial Sequence
  synthetic construct
ctgtgtgatgccaccagatt

SEQ ID NO: 108 - DNA, 21 bp, Artificial Sequence
  synthetic construct
tgaagcaagtttccaagaagc

SEQ ID NO: 109 - DNA, 18 bp, Artificial Sequence
  synthetic construct
ggactgcaccagagccat

SEQ ID NO: 110 - DNA, 20 bp, Artificial Sequence
  synthetic construct
cggctttgaagaagaaagga

SEQ ID NO: 111 - DNA, 16 bp, Artificial Sequence
  synthetic construct
ggaaattttcattatc

SEQ ID NO: 112 - DNA, 16 bp, Artificial Sequence
  synthetic construct
agatattttcatagaa

SEQ ID NO: 113 - DNA, 16 bp, Artificial Sequence
  synthetic construct
gaaaattttcattaaa

SEQ ID NO: 114 - DNA, 16 bp, Artificial Sequence
  synthetic construct
cttctttttcatattt

SEQ ID NO: 115 - DNA, 16 bp, Artificial Sequence
  synthetic construct
tgaatttttcatttta

SEQ ID NO: 116 - DNA, 16 bp, Artificial Sequence
  synthetic construct
tcgatttgtcattctt

SEQ ID NO: 117 - DNA, 16 bp, Artificial Sequence
  synthetic construct
accttatttcatcaat

SEQ ID NO: 118 - DNA, 16 bp, Artificial Sequence
  synthetic construct
gaaaaatatcataaat

SEQ ID NO: 119 - DNA, 16 bp, Artificial Sequence
  synthetic construct
ggaaattttcattttg

SEQ ID NO: 120 - DNA, 16 bp, Artificial Sequence
  synthetic construct
aaattatatcatatcc

SEQ ID NO: 121 - DNA, 16 bp, Artificial Sequence
  synthetic construct
ttaaaatttcattttt

SEQ ID NO: 122 - DNA, 16 bp, Artificial Sequence
  synthetic construct
tattattatcattttc

SEQ ID NO: 123 - DNA, 16 bp, Artificial Sequence
  synthetic construct
aaaaaatttcatatta

SEQ ID NO: 124 - DNA, 16 bp, Artificial Sequence
  synthetic construct
cagttatatcattatc

SEQ ID NO: 125 - DNA, 16 bp, Artificial Sequence
  synthetic construct
cccctatatcattggg

SEQ ID NO: 126 - DNA, 16 bp, Artificial Sequence
  synthetic construct
tggtaatttcatcaag

SEQ ID NO: 127 - DNA, 16 bp, Artificial Sequence
  synthetic construct
ggacaatttcattttc

SEQ ID NO: 128 - DNA, 16 bp, Artificial Sequence
  synthetic construct
tacttttttcattgat

SEQ ID NO: 129 - DNA, 16 bp, Artificial Sequence
  synthetic construct
tcgatttttcatagca

SEQ ID NO: 130 - DNA, 16 bp, Artificial Sequence
  synthetic construct
tatctttgtcatacac

SEQ ID NO: 131 - DNA, 16 bp, Artificial Sequence
  synthetic construct
tggaaatatcattgaa

SEQ ID NO: 132 - DNA, 16 bp, Artificial Sequence
  synthetic construct
atgatttttcatcgat